(19) United States
(12) Patent Application Publication
(10) Pub. No.: US 2021/0339039 A1
(43) Pub. Date: Nov. 4, 2021

(54) RETINAL PHOTOTHERAPY SYSTEM AND METHOD HAVING FIXED PARAMETERS

(71) Applicant: Ojai Retinal Technology, LLC, Los Angeles, CA (US)

(72) Inventor: Jeffrey K. Luttrull, Los Angeles, CA (US)

(73) Assignee: Ojai Retinal Technology, LLC, Los Angeles, CA (US)

(21) Appl. No.: 17/341,666

(22) Filed: Jun. 8, 2021

Related U.S. Application Data
(63) Continuation-in-part of application No. 16/996,308, filed on Aug. 18, 2020, now Pat. No. 11,033,749.
Application No. 16/996,308 is a continuation of application No. 15/918,487, filed on Mar. 12, 2018, now Pat. No. 10,874,873.
Application No. 15/918,487 is a continuation-in-part of application No. 15/460,821, filed on Mar. 16, 2017, now abandoned.
Application No. 15/460,821 is a continuation-in-part of application No. 15/214,726, filed on Jul. 20, 2016, now Pat. No. 10,531,908.
Application No. 15/214,726 is a continuation-in-part of application No. 14/922,885, filed on Oct. 26, 2015, now Pat. No. 9,427,602.
Application No. 14/922,885 is a continuation-in-part of application No. 14/607,959, filed on Jan. 28, 2015, now Pat. No. 9,168,174.
Application No. 14/607,959 is a continuation-in-part of application No. 13/798,523, filed on Mar. 13, 2013, now Pat. No. 10,219,947.
Application No. 13/798,523 is a continuation-in-part of application No. 13/481,124, filed on May 25, 2012, now Pat. No. 9,381,115.
Application No. 15/918,487 is a continuation-in-part of application No. 15/629,002, filed on Jun. 21, 2017, now Pat. No. 10,278,863.
Application No. 15/629,002 is a continuation-in-part of application No. 15/583,096, filed on May 1, 2017, now Pat. No. 10,953,241.
Application No. 15/583,096 is a continuation-in-part of application No. 15/232,320, filed on Aug. 9, 2016, now Pat. No. 9,962,291.
Application No. 15/232,320 is a continuation-in-part of application No. 15/178,842, filed on Jun. 10, 2016, now Pat. No. 9,626,445.
Application No. 15/178,842 is a continuation-in-part of application No. 14/921,890, filed on Oct. 23, 2015, now Pat. No. 9,381,116.
Application No. 14/921,890 is a continuation-in-part of application No. 14/607,959, filed on Jan. 28, 2015, now Pat. No. 9,168,174.
Application No. 14/607,959 is a continuation-in-part of application No. 13/798,523, filed on Mar. 13, 2013, now Pat. No. 10,219,947.
Application No. 13/798,523 is a continuation-in-part of application No. 13/481,124, filed on May 25, 2012, now Pat. No. 9,381,115.

(51) Int. Cl. A61N 5/06; A61F 9/008; A61N 7/02; A61N 5/04; A61N 5/02; A61N 5/00

(52) U.S. Cl. CPC A61N 5/0603; A61F 9/00817; A61N 7/022; A61N 5/0625; A61N 5/045; A61N 1/403; A61F 9/00821; A61F 9/008; A61N 5/00; A61N 2005/0608; A61N 5/025

(57) ABSTRACT

A retinal phototherapy or photostimulation system includes a laser console generating at least one treatment beam having parameters to photostimulate or treat, while not permanently damaging, a retinal target tissue. The parameters of the at least one treatment beam are fixed so as not to be alterable by a medical provider. A projector or camera projects the at least one treatment beam onto at least a portion of the retina, and a scanning mechanism controllably directs the at least one treatment beam to treatment areas of the retina.

### RELATED APPLICATIONS

[Para 1] This application is a continuation-in-part of U.S. application Ser. No. 16/966,308, filed on Aug. 18, 2020, which is a continuation of Application Ser. No. 15/918,487, filed on Mar. 12, 2018, which is a continuation-in-part of application Ser. No. 15/460,821, filed on Mar. 16, 2017, now abandoned, which is a continuation-in-part of application Ser. No. 15/214,726, filed on Jul. 20, 2016, now U.S. Pat. No. 10,531,908, which is a continuation-in-part of application Ser. No. 14/922,885, filed on Oct. 26, 2015, now U.S. Pat. No. 9,427,602, which is a continua-tion-in-part of application Ser. No. 14/607,959, filed on Jan. 28, 2015, now U.S. Pat. No. 9,168,174, which is a continuation-in-part of application Ser. No. 13/798,523, filed on Mar. 13, 2013, now U.S. Pat. No. 10,219,947, which is a continuation-in-part of application Ser. No. 13/481,124, filed on May 25, 2012, now U.S. Pat. No. 9,381,115, said application Ser. No. 15/918,487 is a con-tinuation-in-part of application Ser. No. 15/629,002, filed on Jun. 21, 2017, now U.S. Pat. No. 10,278,863, which is a continuation-in-part of application Ser. No. 15/583,096, filed on May 1, 2017, which is a continuation-in-part of application Ser. No. 15/232,320, filed on Aug. 9, 2016, now U.S. Pat. No. 9,962,291, which is a continuation-in-part of application Ser. No. 15/178,842, filed on Jun. 10, 2016, now U.S. Pat. No. 9,626,445, which is a continuation-in-part of application Ser. No. 14/921,890, filed on Oct. 23, 2015, now U.S. Pat. No. 9,381,116, which is a continuation-in-part of application Ser. No. 14/607,959, filed on Jan. 28, 2015, now U.S. Pat. No. 9,168,174, which is a continuation-in-part of application Ser. No. 13/798, 523, filed on Mar. 13, 2013, now U.S. Pat. No. 10,219,947, which is a continuation-in-part of application Ser. No. 13/481,124, filed on May 25, 2012, now U.S. Pat. No. 9,381,115.

### BACKGROUND OF THE INVENTION

[Para 2] The present invention is generally directed to retinal phototherapy or photostimulation. More particularly, the present invention is directed to a retinal phototherapy or photostimulation system and method wherein parameters of one or more treatment beams are selected and fixed so as not to be alterable by a medical provider to ensure the retinal target tissue is photostimulated or treated while not permanently damaging the retinal tissue.

[Para 3] Until the advent of thermal retinal photocoagulation, there was generally no effective treatment for many retinal diseases, such as diabetic retinopathy. With visible end point photocoagulation, laser light absorption heats pigmented tissue at the laser sight. Heat conduction spreads this temperature increase from the retinal pigment epithelium (RPE) and choroid to overlying non-pigmented and adjacent unexposed tissues. Laser lesions become visible so as to track the treated areas. In fact, it has been believed that actual tissue damage and scarring are necessary in order to create the benefits of traditional photocoagulation. Photocoagulation has been found to be an effective means of producing retinal scars, it has become the technical standard for treatment of various retinal diseases, including macular photocoagulation for diabetic macular edema and other retinal diseases for decades.

[Para 4] That Iatrogenic retinal damage is necessary for effective laser treatment of retinal vascular disease has been universally accepted for almost five decades, and remains the prevailing notion. Although providing a clear advantage compared to no treatment, current retinal photocoagulation treatments, which produce visible gray to white retinal burns and scarring, have disadvantages and drawbacks. Conventional photocoagulation is often painful. Local anesthesia, with its own attendant risks, may be required. Alternatively, treatment may be divided into stages over an extended period of time to minimize treatment pain and post-operative inflammation. Transient reduction and visual acuity is common following conventional photocoagulation. In fact, thermal tissue damage may be the sole source of the many potential complications of conventional photocoagulation which may lead to immediate and late visual loss.

[Para 5] The fovea/macula region is a portion of the eye used for color vision and fine detail vision. The fovea is at the center of the macula, where the concentration of the cells needed for central vision is the highest. Although it is the area where diseases such as age-related macular degeneration are so damaging, this is the area where conventional photocoagulation phototherapy cannot be used as damaging the cells in the foveal area can significantly damage the patient's vision. Thus, with current conventional photocoagulation therapies, the foveal region is avoided.

[Para 6] Recently, the inventors discovered that subthreshold photocoagulation in which no visible damage or laser lesions, or even any which are detectable by any known means including ophthalmoscopy, infrared, color, red-free or autofluorescence fundus photography and standard retro-mode, intravenous fundus fluorescein or indocyanine green angiographically, or spectral-domain optical coherence tomography at the time of treatment or any time thereafter, has produced similar beneficial results in treatments as traditional tissue damaging photocoagulation without many of the drawbacks and complications thereof.

[Para 7] It is believed that a possible mechanism caused by such subthreshold photocoagulation is the raising of the temperature of the tissue in such a controlled manner to selectively stimulate heat shock protein activation and/or production and facilitation of protein repair, which serves as a mechanism for therapeutically treating the tissue. It is believed that this micropulse train thermally activates heat shock proteins (HSPs) in the targeted tissue. In the case of retinal tissue, the process thermally activates HSPs in the RPE layer immediately behind the retinal layer containing the visually sensitive rods and cones, and these activated HSPs then reset the diseased retina to its healthy condition by removing the repairing damaged proteins. This then results in improved RPE function, improves retinal function and autoregulation, restorative acute inflammation, reduced chronic inflammation, and systematic immunodulation. The laser-triggered effects then slow, stop or reverse retinal disease, improve visual function and reduce the risk of visual loss.

[Para 8] It has been determined that with the proper operating parameters, subthreshold photocoagulation treatment can be, and may ideally be, applied to the entire retina, including sensitive areas such as the fovea, without visible tissue damage or the resulting drawbacks or complications of conventional visible retinal photocoagulation treatments. It is believed that raising tissue temperature in such a controlled manner to selectively stimulate heat shock protein activation has benefits in other tissues as well. The selection of the proper operating parameters, including wavelength, power, pulse train duration and duty cycle of the laser treatment beam is important to provide the therapeutic or stimulation benefit while not damaging the retinal tissue to which the laser light is applied. Selecting and using laser beam generation and application parameters outside of the acceptable ranges can result in damage to the patient's retina. Moreover, improperly selecting the combination of laser treatment light beam parameters, even within the safely-determined parameters, can result in less than ideal treatment or photostimulation.

[Para 9] Accordingly, there is a continuing need for a retinal phototherapy or photostimulation system which generates one or more laser treatment beams having operating and application parameters selected so as to treat or photostimulate retinal tissue while not permanently damaging the retinal tissue. What is also needed is such a system in which optimal parameters are selected and are fixed and not alterable, such as by a medical provider, after being set so as to ensure that the treatment beam is both effective and safe. The present invention fulfills these needs and provides other related advantages.

### SUMMARY OF THE INVENTION

[Para 10] The present invention resides in a retinal phototherapy or photostimulation system and related method. The system generally comprises a laser console generating at least one pulsed treatment beam. The at least one treatment beam has parameters of wavelength, power, pulse train duration and duty cycle to photostimulate or treat a retinal target tissue while not permanently damaging the retinal target tissue. The parameters of the at least one treatment beam are fixed so as not to be alterable by a medical provider.

[Para 11] The at least one treatment beam has a wavelength between 530 nm to 1300 nm, a duty cycle of less than 10%, and a pulse train duration between 0.1 and 0.6 seconds. The at least one treatment beam may have a wavelength between 750 nm and 850 nm, a duty cycle between 2% and 5%, and a pulse train duration of between 0.15 and 0.5 seconds. The laser console has a power output of between 1.0-3.0 watts.

[Para 12] A projector or camera projects the at least one treatment beam onto at least a portion of a retina. The at least one treatment beam may create a treatment spot on the retina having a size of between 100-1000 micrometers.

[Para 13] A scanning mechanism controllably directs the at least one treatment beam to treatment areas of the retina. The at least one treatment beam may be applied to a plurality of target tissue areas, wherein adjacent target tissue areas are separated to avoid thermal tissue damage.

[Para 14] The at least one treatment beam stimulates heat shock protein activation in the target tissue. The at least one treatment beam raises a target tissue temperature to a desired level while maintaining an average temperature rise of the target tissue over a period of time at or below a predetermined level so as not to permanently damage the target tissue. The at least one treatment beam may raise a target tissue temperature to no greater than 11° C. to achieve a therapeutic or prophylactic effect. The at least one treatment beam may raise the target tissue temperature between 6° C. to 11° C. at least during application of the pulse energy source to the target tissue. The average temperature rise of the target tissue over several minutes is maintained at or below a predetermined level so as not to permanently damage the target tissue. The average temperature rise of the target tissue over several minutes, such as six minutes or less, may be maintained at 1° C. or less.

[Para 15] Other features and advantages of the present invention will become apparent from the following more detailed description, taken in conjunction with the accompanying drawings, which illustrate, by way of example, the principles of the invention.

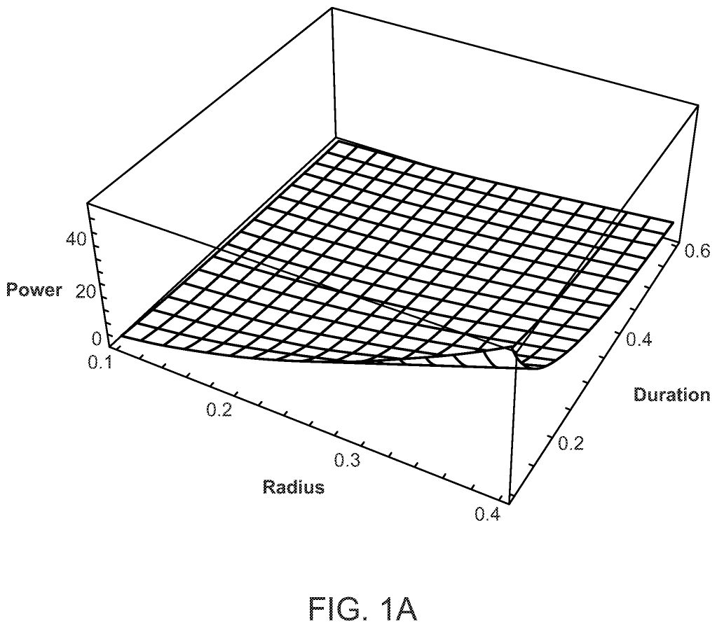

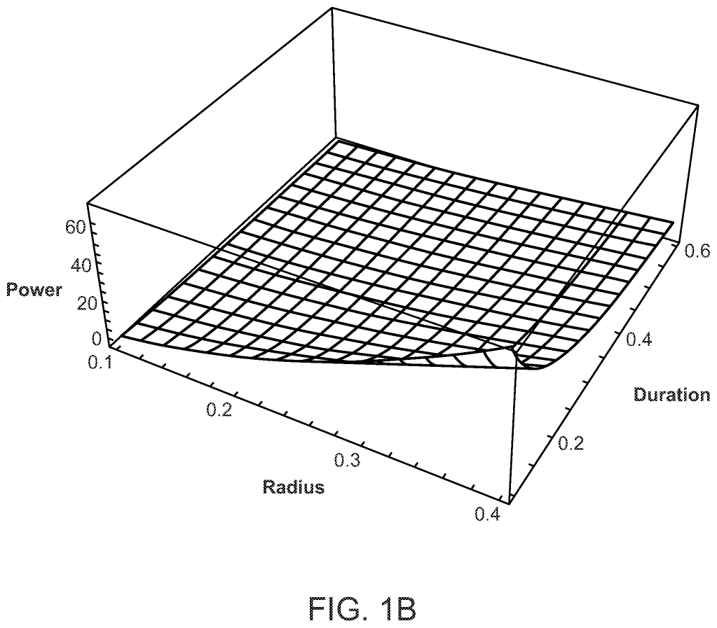

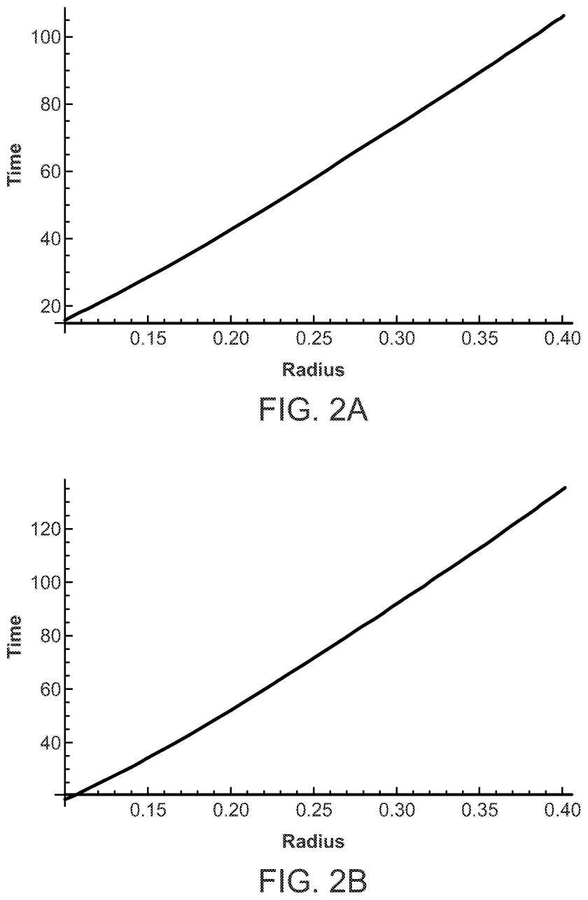

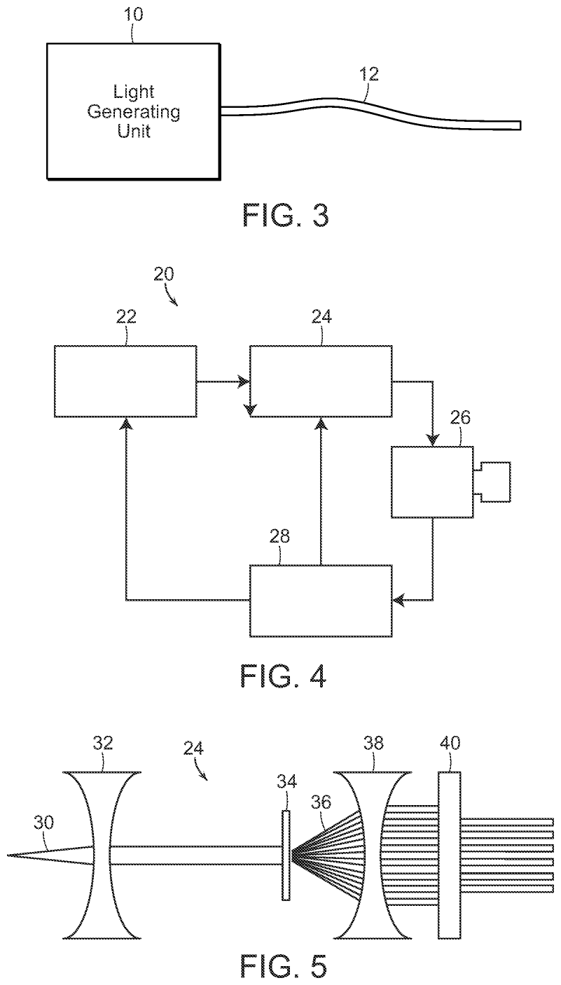

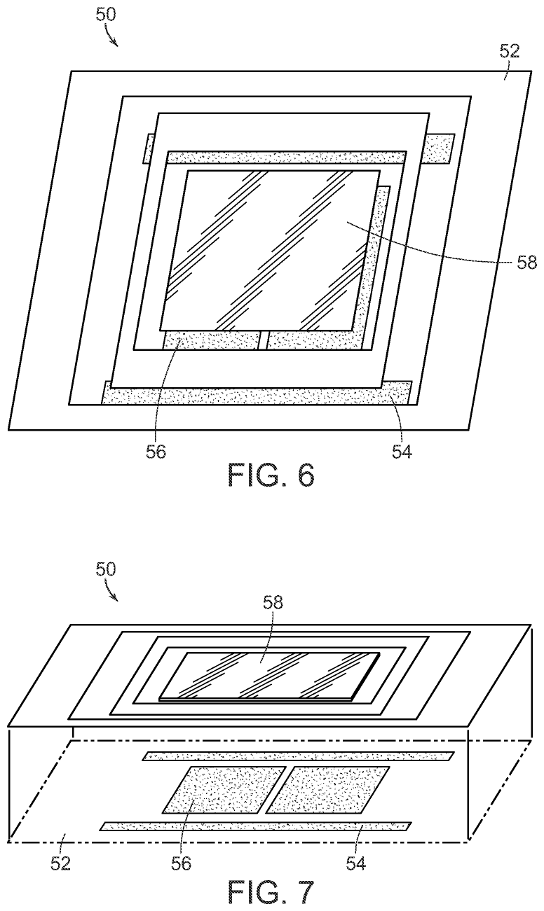

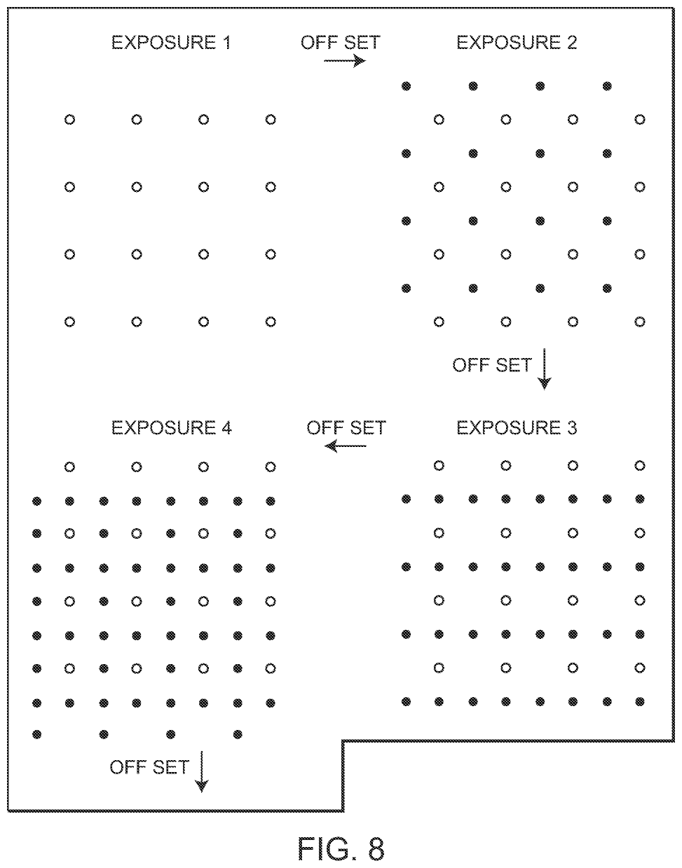

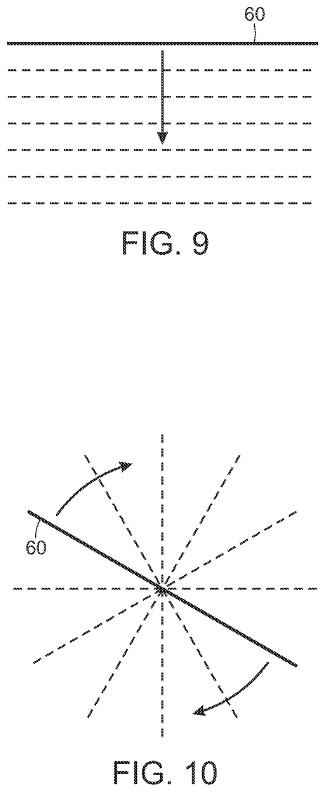

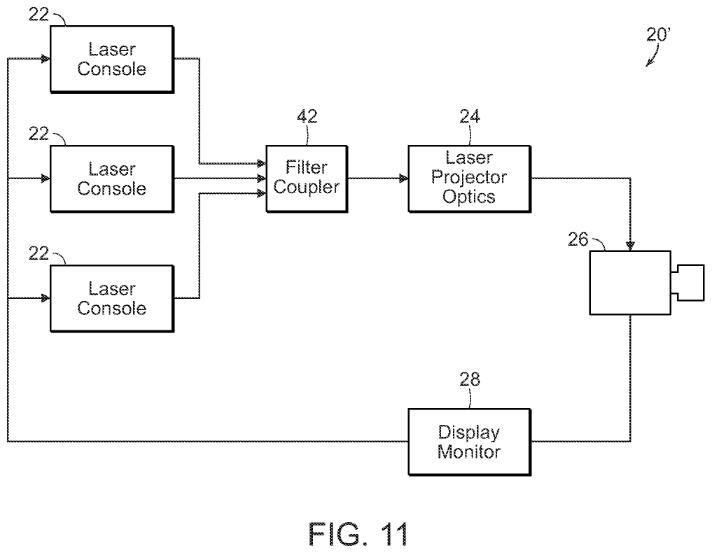

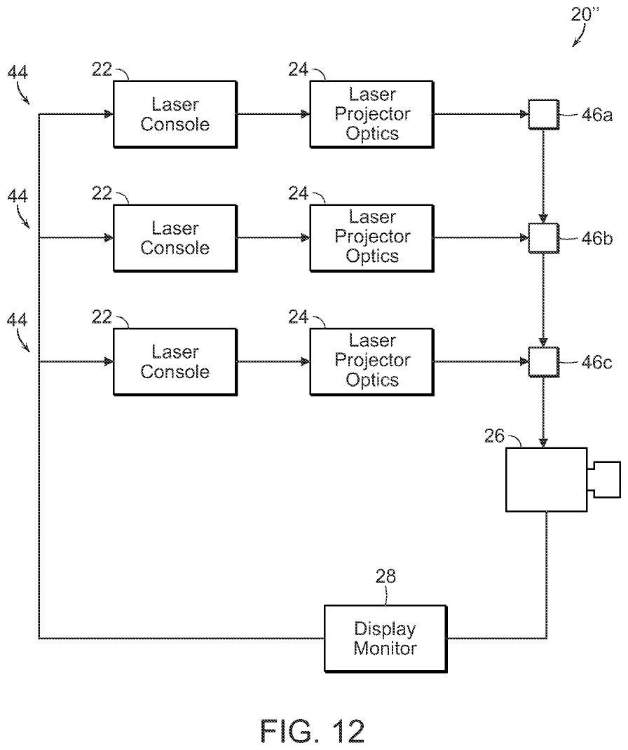

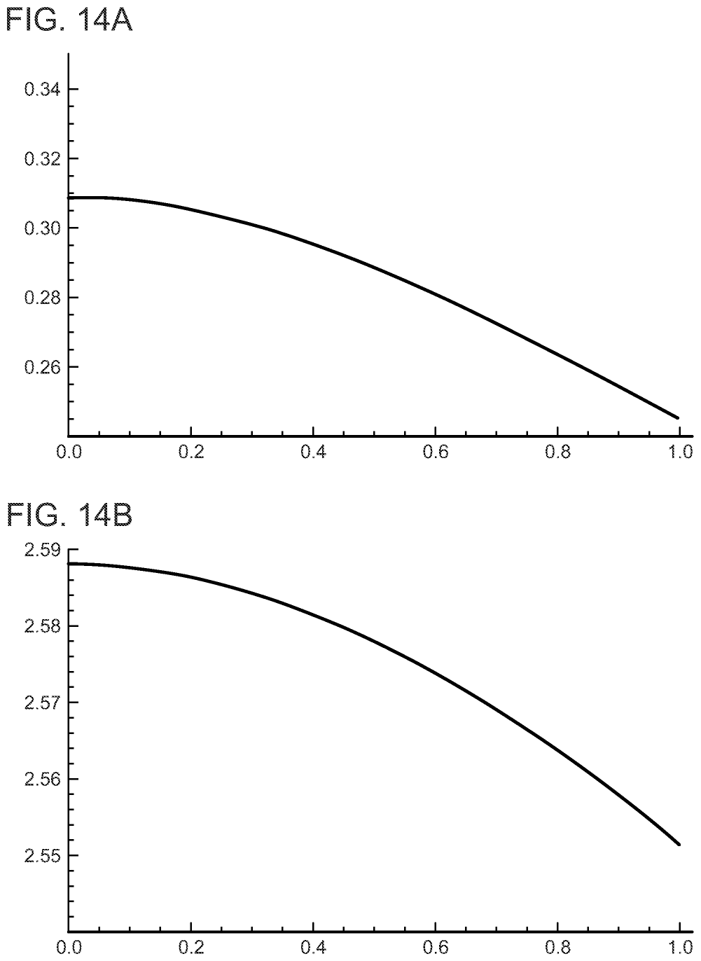

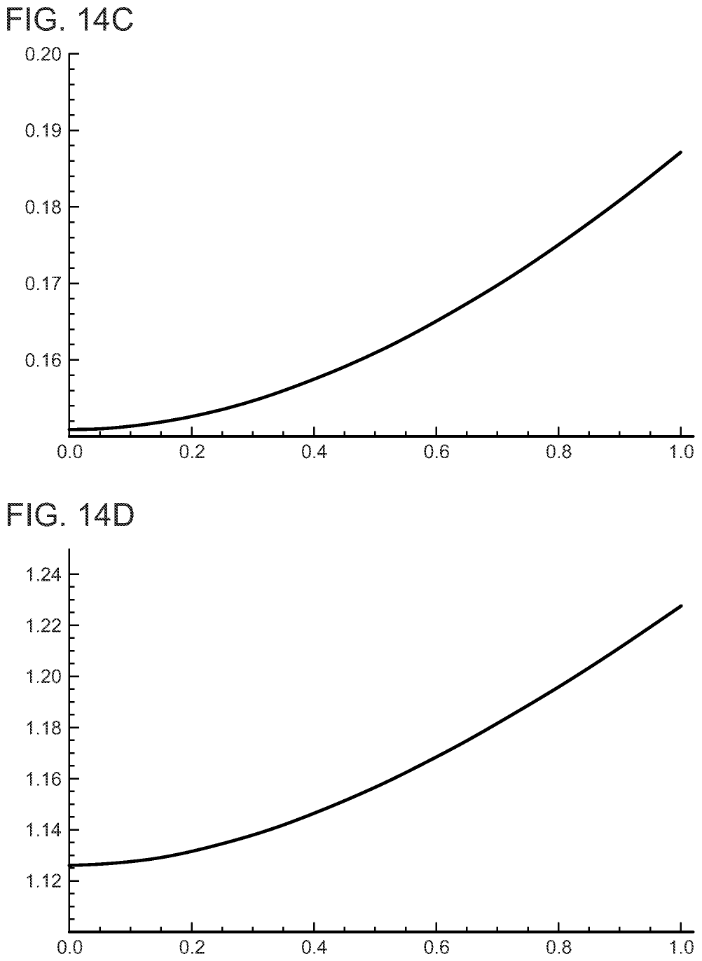

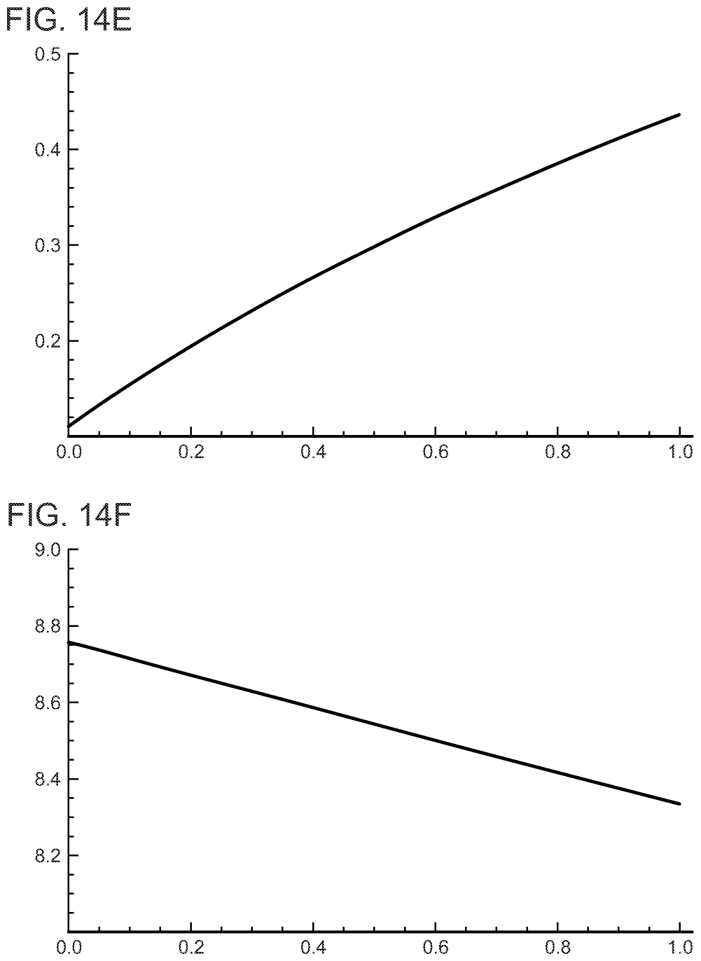

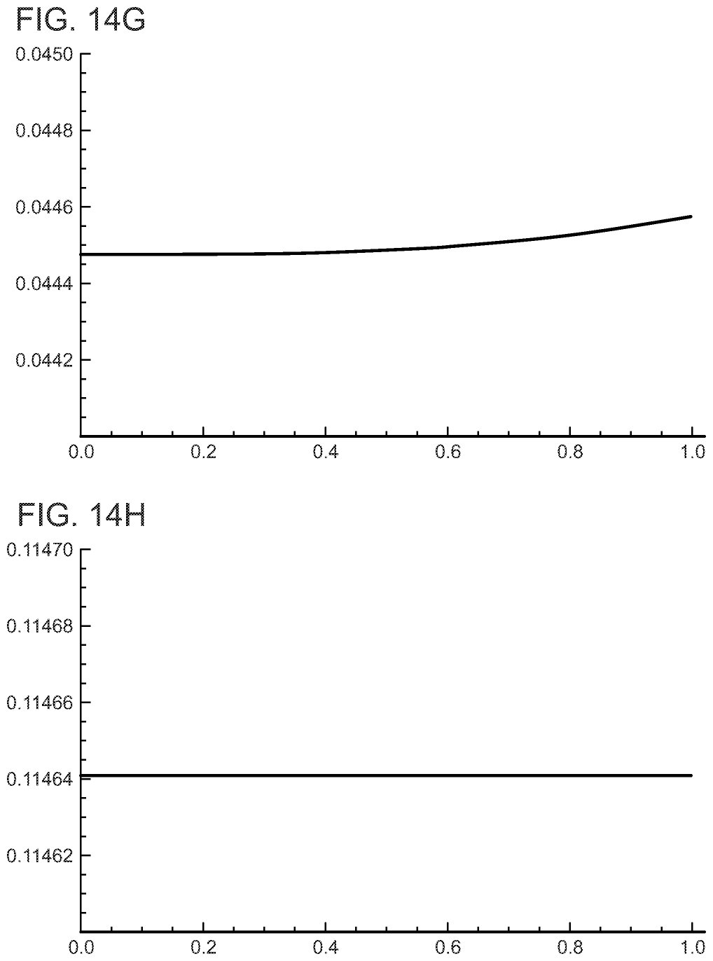

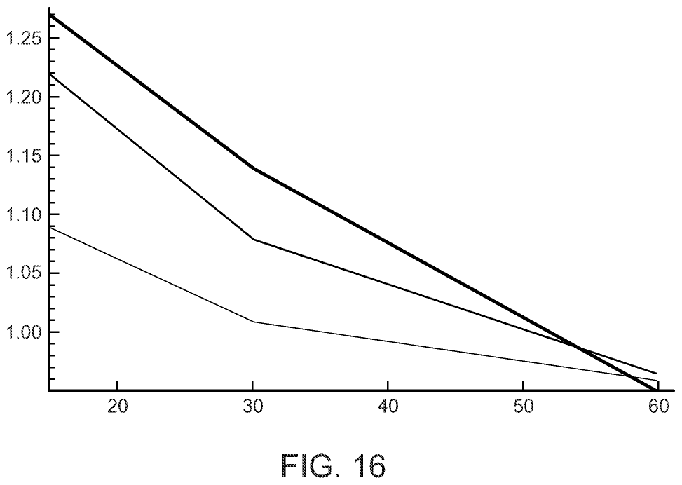

### BRIEF DESCRIPTION OF THE DRAWINGS

[Para 16] The accompanying drawings illustrate the invention. In such drawings:

[Para 1 7] FIGS. 1 A and 1B are graphs illustrating the average power of a laser source compared to a source radius and pulse train duration of the laser;

[Para 18] FIGS. 2A and 2B are graphs illustrating the time for the temperature to decay depending upon the laser source radius and wavelength;

[Para 19] FIG. 3 is a diagrammatic view of a light generating unit that produces timed series of pulses, having a light pipe extending therefrom, in accordance with the present invention;

[Para 20] FIG. 4 is a diagrammatic view illustrating a system used to generate a treatment laser light beam, in accordance with the present invention;

[Para 21] FIG. 5 is a diagrammatic view of optics used to generate a laser light geometric pattern, in accordance with the present invention;

[Para 22] FIG. 6 is a top plan view of an optical scanning mechanism, used in accordance with the present invention;

[Para 23] FIG. 7 is a partially exploded view of the optical scanning mechanism of FIG. 6, illustrating the various component parts thereof;

[Para 24] FIG. 8 illustrates controlled offsets of exposure of an exemplary geometric pattern grid of laser spots to treat the target tissue, in accordance with an embodiment of the present invention;

[Para 25] FIG. 9 is a diagrammatic view illustrating the use of a geometric object in the form of a line controllably scanned to treat an area of the target tissue;

[Para 26] FIG. 10 is a diagrammatic view similar to FIG. 9, but illustrating the geometric line or bar rotated to treat the target tissue;

[Para 27] FIG. 11 is a diagrammatic view illustrating an alternate embodiment of the system used to generate treatment laser light beams for treating tissue, in accordance with the present invention;

[Para 28] FIG. 12 is a diagrammatic view illustrating yet another embodiment of a system used to generate treatment laser light beams to treat tissue in accordance with the present invention;

[Para 29] FIGS. 1 3A and 13B are graphs depicting the behavior of HSP cellular system components over time following a sudden increase in temperature;

[Para 30] FIGS. 14A-14H are graphs depicting the behavior of HSP cellular system components in the first minute following a sudden increase in temperature;

[Para 31] FIGS. 1 5A and 15B are graphs illustrating variation in the activated concentrations of HSP and unactivated HSP in the cytoplasmic reservoir over an interval of one minute, in accordance with the present invention; and

[Para 32] FIG. 16 is a graph depicting the improvement ratios versus interval between treatments, in accordance with the present invention.

### DETAILED DESCRIPTION OF THE PREFERRED EMBODIMENTS

[Para 33] As shown in the accompanying drawings, and as more fully described herein, the present invention is directed to a system and method for delivering a pulsed energy, such as one or more light beams having energy parameters selected to cause a thermal time-course in tissue to raise the tissue temperature over a short period of time to a sufficient level to achieve a therapeutic effect while maintaining an average tissue temperature over a prolonged period of time below a predetermined level so as to avoid permanent tissue damage. It is believed that the creation of the thermal time-course stimulates heat shock protein activation or production and facilitates protein repair without causing any damage.

[Para 34] The inventors have discovered that electromagnetic radiation can be applied to retinal tissue in a manner that does not destroy or damage the retinal tissue while achieving beneficial effects on eye diseases. More particularly, a laser light beam can be generated that is therapeutic, yet sublethal to retinal tissue cells and thus avoids damaging photocoagulation in the retinal tissue which provides preventative and protective treatment of the retinal tissue of the eye. It is believed that this may be due, at least in part, to the stimulation and activation of heat shock proteins and the facilitation of protein repair in the retinal tissue.

[Para 35] Various parameters of the light beam must be taken into account and selected so that the combination of the selected parameters achieve the therapeutic effect while not permanently damaging the tissue. These parameters include laser wavelength, average laser power, total pulse duration, and duty cycle of the pulse train.

[Para 36] The selection of these parameters may be determined by requiring that the Arrhenius integral for HSP activation be greater than 1 or unity. Arrhenius integrals are used for analyzing the impacts of actions on biological tissue. At the same time, the selected parameters must not permanently damage the tissue. Thus, the Arrhenius integral for damage may also be used, wherein the solved Arrhenius integral is less than 1 or unity.

[Para 37] Alternatively, the FDA/FCC constraints on energy deposition per unit gram of tissue and temperature rise as measured over periods of minutes be satisfied so as to avoid permanent tissue damage. Generally speaking, tissue temperature rises of between 6° C. and 11° C. can create therapeutic effect, such as by activating heat shock proteins, whereas maintaining the average tissue temperature over a prolonged period of time, such as over several minutes, such as six minutes, below a predetermined temperature, such as 6° C. and even 1° C. or less in certain circumstances, will not permanently damage the tissue.

[Para 38] The inventors have discovered that generating a subthreshold, sublethal micropulse laser light beam which has a wavelength greater than 530 nm and a duty cycle of less than 10% at a predetermined intensity or power and a predetermined pulse length or exposure time creates desirable retinal photostimulation without any visible burn areas or tissue destruction. More particularly, a laser light beam having a wavelength of between 530 nm to 1300 nm, and in a particularly preferred embodiment between 750 nm and 850 nm, having a duty cycle of approximately 2%-5%, a pulse train duration of 0.15 to 0.5 seconds, and a predetermined intensity or power (such as between 100-590 watts per square centimeter at the retina or approximately 1 watt per laser spot for each treatment spot at the retina) and a predetermined pulse length or exposure time (such as between 100 and 600 milliseconds) creates a sublethal, “true subthreshold” retinal photostimulation in which all areas of the retinal pigment epithelium exposed to the laser irradiation are preserved and available to contribute therapeutically. In other words, the inventors have found that raising the retinal tissue at least up to a therapeutic level but below a cellular or tissue lethal level creates the benefit of the prior art methods without destroying, burning or otherwise damaging the retinal tissue. This is referred to herein as subthreshold diode micropulse laser treatment (SDM).

[Para 39] SDM does not produce laser-induced retinal damage (photocoagulation), and has no known adverse treatment effect, and has been reported to be an effective treatment in a number of retinal disorders (including diabetic macular edema (DME) proliferative diabetic retinopathy (PDR), macular edema due to branch retinal vein occlusion (BRVO), central serous chorioretinopathy (CSR), reversal of drug tolerance, and prophylactic treatment of progressive degenerative retinopathies such as dry age-related macular degeneration, Stargardts' disease, cone dystrophies, and retinitis pigmentosa. The safety of SDM is such that it may be used transfoveally in eyes with 20/20 visual acuity to reduce the risk of visual loss due to early fovea-involving DME.

[Para 40] A mechanism through which SDM might work is the generation or activation of heat shock proteins (HSPs). Despite a near infinite variety of possible cellular abnormalities, cells of all types share a common and highly conserved mechanism of repair: heat shock proteins (HSPs). HSPs are elicited almost immediately, in seconds to minutes, by almost any type of cell stress or injury. In the absence of lethal cell injury, HSPs are extremely effective at repairing and returning the viable cell toward a more normal functional state. Although HSPs are transient, generally peaking in hours and persisting for a few days, their effects may be long lasting. HSPs reduce inflammation, a common factor in many disorders.

[Para 41] Laser treatment can induce HSP production or activation and alter cytokine expression. The more sudden and severe the non-lethal cellular stress (such as laser irradiation), the more rapid and robust HSP activation. Thus, a burst of repetitive low temperature thermal spikes at a very steep rate of change (−7° C. elevation with each 100 μs micropulse, or 70,000° C/sec) produced by each SDM exposure is especially effective in stimulating activation of HSPs, particularly compared to non-lethal exposure to sub-threshold treatment with continuous wave lasers, which can duplicate only the low average tissue temperature rise.

[Para 42] Laser wavelengths below 530 nm produce increasingly cytotoxic photochemical effects. Between 530 nm and 1310 nm and particularly around 810 nm, SDM produces photothermal, rather than photochemical, cellular stress. Thus, SDM is able to affect the tissue without damaging it. The clinical benefits of SDM are thus primarily produced by sub-morbid photothermal cellular HSP activation. In dysfunctional cells, HSP stimulation by SDM results in normalized cytokine expression, and consequently improved structure and function. The therapeutic effects of this “low-intensity” laser/tissue interaction are then amplified by “high-density” laser application, recruiting all the dysfunctional cells in the targeted tissue area by densely/confluently treating a large tissue area, including all areas of pathology, thereby maximizing the treatment effect. These principles define the treatment strategy of SDM described herein.

[Para 43] Because normally functioning cells are not in need of repair, HSP stimulation in normal cells would tend to have no notable clinical effect. The “patho-selectivity” of near infrared laser effects, such as SDM, affecting sick cells but not affecting normal ones, on various cell types is consistent with clinical observations of SDM. SDM has been reported to have a clinically broad therapeutic range, unique among retinal laser modalities, consistent with American National Standards Institute “Maximum Permissible Exposure” predictions. While SDM may cause direct photothermal effects such as entropic protein unfolding and disaggregation, SDM appears optimized for clinically safe and effective stimulation of HSP-mediated repair.

[Para 44] As noted above, while SDM stimulation of HSPs is non-specific with regard to the disease process, the result of HSP mediated repair is by its nature specific to the state of the dysfunction. HSPs tend to fix what is wrong, whatever that might be. Thus, the observed effectiveness of SDM in retinal conditions as widely disparate as BRVO, DME, PDR, CSR, age-related and genetic retinopathies, and drug-tolerant NAMD. Conceptually, this facility can be considered a sort of “Reset to Default” mode of SDM action. For the wide range of disorders in which cellular function is critical, SDM normalizes cellular function by triggering a “reset” (to the “factory default settings”) via HSP-mediated cellular repair.

[Para 45] The inventors have found that SDM treatment of patients suffering from age-related macular degeneration (AMD) can slow the progress or even stop the progression of AMD. Most of the patients have seen significant improvement in dynamic functional log MAR mesoptic visual acuity and mesoptic contrast visual acuity after the SDM treatment. It is believed that SDM works by targeting, preserving, and “normalizing” (moving toward normal) function of the retinal pigment epithelium (RPE).

[Para 46] SDM has also been shown to stop or reverse the manifestations of the diabetic retinopathy disease state with-out treatment-associated damage or adverse effects, despite the persistence of systemic diabetes mellitus. On this basis it is hypothesized that SDM might work by inducing a return to more normal cell function and cytokine expression in diabetes-affected RPE cells, analogous to hitting the “reset” button of an electronic device to restore the factory default settings. Based on the above information and studies, SDM treatment may directly affect cytokine expression via heat shock protein (HSP) activation in the targeted tissue.

[Para 47] As indicated above, subthreshold diode micropulse laser (SDM) photostimulation has been effective in stimulating direct repair of slightly misfolded proteins in eye tissue. Besides HSP activation, another way this may occur is because the spikes in temperature caused by the micro-pulses in the form of a thermal time-course allows diffusion of water inside proteins, and this allows breakage of the peptide-peptide hydrogen bonds that prevent the protein from returning to its native state. The diffusion of water into proteins results in an increase in the number of restraining hydrogen bonds by a factor on the order of a thousand. Thus, it is believed that this process could be applied to other tissues and diseases advantageously as well.

[Para 48] As explained above, the energy source to be applied to the target tissue will have energy and operating parameters which must be determined and selected so as to achieve the therapeutic effect while not permanently damaging the tissue. Using a light beam energy source, such as a laser light beam, as an example, the laser wavelength, duty cycle and total pulse train duration parameters must be taken into account. Other parameters which can be considered include the radius of the laser source as well as the average laser power. Adjusting or selecting one of these parameters can have an effect on at least one other parameter.

[Para 49] Thus, in accordance with the present invention, to maintain safety and prevent user error, a retinal phototherapy or photostimulation system embodying the present invention has the at least one treatment parameters, including wavelength, power, pulse train duration and duty cycle, preselected and fixed so as not to be alterable by a medical provider or other end user. In this manner, the pulsed energy generator, such as a laser console, will generate a pulsed energy source, such as a pulsed treatment laser light beam, having a combination of parameters selected so as to photostimulate and/or treat the retinal tissue without destroying, permanently damaging, or even damaging the retinal tissue whatsoever. As the adjusting or selecting of one parameter can have effect on at least one of the other parameters, the proper or most beneficial preselected parameters of wavelength, power, pulse train duration and duty cycle of the at least one treatment beam may be selected and fixed before delivery to the medical care provider or other end user. The combination of preselected parameters will be safe and can be optimized. As the medical provider or other end user cannot alter the fixed operating parameters, there is no chance of user error or inadvertently creating a combination of light treatment beam operating parameters which could cause damage to the retinal tissue.

[Para 50] For example, the at least one treatment beam may have a wavelength selected between 530 nm to 1300 nm, a duty cycle of less than 10% and a pulse train duration of between 0.1 and 0.6 seconds. Within the foregoing ranges of wavelength, the duty cycle may be between 2% and 5%. The peak laser power output may be between 0.5 and 3.0 watts. A treatment spot size on the retinal tissue formed by the one or more treatment beams may be between 100-1000 micrometers. More particularly, the at least one treatment beam may have a wavelength between 750 nm and 850 nm, a duty cycle between 2% and 5%, and a pulse train duration of between 0.15 and 0.5 seconds. As an example, the system may have the fixed parameters of 810 nm, a duty cycle of 5%, a pulse train duration of 0.3 seconds, a power of 1.8 watts, and a resulting spot size of 500 micrometers. An acceptable alternative for a wavelength of approximately 810 nm would be a duty cycle of 2%, a pulse train duration of 0.5 seconds, a peak laser power output of 2.19 watts, and a resulting retinal treatment spot of 400 micrometers. Other specific combinations within the ranges given above are also possible. However, preselecting and fixing these operating parameters would prevent an end user, such as a medical care provider, from adjusting any of the parameters to outside of an acceptable range which might cause damage or not provide effective treatment.

[Para 51 ] FIGS. 1A and 1B illustrate graphs showing the average power in watts as compared to the laser source radius (between 0.1 cm and 0.4 cm) and pulse train duration (between 0.1 and 0.6 seconds). FIG. 1A shows a wavelength of 880 nm, whereas FIG. 1B has a wavelength of 1000 nm. It can be seen in these figures that the required power decreases monotonically as the radius of the source decreases, as the total train duration increases, and as the wavelength decreases. The preferred parameters for the radius of the laser source is 1 mm-4 mm. For a wavelength of 880 nm, the minimum value of power is 0.55 watts, with a radius of the laser source being 1 mm, and the total pulse train duration being 600 milliseconds. The maximum value of power for the 880 nm wavelength is 52.6 watts when the laser source radius is 4 mm and the total pulse drain duration is 100 milliseconds. However, when selecting a laser having a wavelength of 1000 nm, the minimum power value is 0.77 watts with a laser source radius of 1 mm and a total pulse train duration of 600 milliseconds, and a maximum power value of 73.6 watts when the laser source radius is 4 mm and the total pulse duration is 100 milliseconds. The corresponding peak powers, during an individual pulse, are obtained from the average powers by dividing by the duty cycle.

[Para 52] The volume of the tissue region to be heated is determined by the wavelength, the absorption length in the relevant tissue, and by the beam width. The total pulse duration and the average laser power determine the total energy delivered to heat up the tissue, and the duty cycle of the pulse train gives the associated spike, or peak, power associated with the average laser power. Preferably, the pulsed energy source energy parameters are selected so that approximately 20 to 40 joules of energy is absorbed by each cubic centimeter of the target tissue. The absorption length is very small in the thin melanin layer in the retinal pigmented epithelium.

[Para 53] It has been determined that the target tissue can be heated to up to approximately 11° C. for a short period of time, such as less than one second, to create the therapeutic effect of the invention while maintaining the target tissue average temperature to a lower temperature range, such as less than 6° C. or even 1° C. or less over a prolonged period of time, such as several minutes. The selection of the duty cycle and the total pulse train duration provide time intervals in which the heat can dissipate. A duty cycle of less than 10%, and preferably between 2.5% and 5%, with a total pulse duration of between 100 milliseconds and 600 milliseconds has been found to be effective. FIGS. 2A and 2B illustrate the time to decay from 10° C. to 1° C. for a laser source having a radius of between 0.1 cm and 0.4 cm with the wavelength being 880 nm in FIG. 2A and 1000 nm in FIG. 2B. It can be seen that the time to decay is less when using a wavelength of 880 nm, but either wavelength falls within the acceptable requirements and operating param-eters to achieve the benefits of the present invention while not causing permanent tissue damage.

[Para 54] It has been found that the average temperature rise of the desired target region increasing at least 6° C. and up to 11° C., and preferably approximately 10° C., during the total irradiation period results in HSP activation. The control of the target tissue temperature is determined by choosing source and target parameters such that the Arrhenius integral for HSP activation is larger than 1, while at the same time assuring compliance with the conservative FDA/FCC requirements for avoiding damage or a damage Arrhenius integral being less than 1.

[Para 55] In order to meet the conservative FDA/FCC constraints to avoid permanent tissue damage, for light beams and other electromagnetic radiation sources, the average temperature rise of the target tissue over any six-minute period is 1° C. or less. FIGS. 2A and 2B above illustrate the typical decay times required for the temperature in the heated target region to decrease by thermal diffusion from a temperature rise of approximately 10° C. to 1° C. as can be seen in FIG. 2A when the wavelength is 880 nm and the source diameter is 1 millimeter, the temperature decay time is 16 seconds. The temperature decay time is 107 seconds when the source diameter is 4 mm. As shown in FIG. 2B, when the wavelength is 1000 nm, the temperature decay time is 18 seconds when the source diameter is 1 mm and 136 seconds when the source diameter is 4 mm. This is well within the time of the average temperature rise being maintained over the course of several minutes, such as 6 minutes or less. While the target tissue's temperature is raised, such as to approximately 10° C., very quickly, such as in a fraction of a second during the application of the energy source to the tissue, the relatively low duty cycle provides relatively long periods of time between the pulses of energy applied to the tissue and the relatively short pulse train duration ensure sufficient temperature diffusion and decay within a relatively short period of time comprising several minutes, such as 6 minutes or less, that there is no permanent tissue damage.

[Para 56] The pulse train mode of energy delivery has a distinct advantage over a single pulse or gradual mode of energy delivery, as far as the activation of remedial HSPs and the facilitation of protein repair is concerned. There are two considerations that enter into this advantage:

[Para 57] First, a big advantage for HSP activation and protein repair in an SDM energy delivery mode comes from producing a spike temperature of the order of 10° C. This large rise in temperature has a big impact on the Arrhenius integrals that describe quantitatively the number of HSPs that are activated and the rate of water diffusion into the proteins that facilitates protein repair. This is because the temperature enters into an exponential that has a big amplification effect.

[Para 58] It is important that the temperature rise not remain at the high value (10° C. or more) for long, because then it would violate the FDA and FCC requirements that over periods of minutes the average temperature rise must be less than 1° C.

[Para 59] An SDM mode of energy delivery uniquely satisfies both of these foregoing considerations by judicious choice of the power, pulse time, pulse interval, and the volume of the target region to be treated. The volume of the treatment region enters because the temperature must decay from its high value of the order of 10° C. fairly rapidly in order for the long term average temperature rise not to exceed the long term FDA/FCC limit of 1° C. or less for electromagnetic radiation energy sources.

[Para 60] With reference now to FIG. 3, a light generating unit 10, such as a laser having a desired wavelength and/or frequencies and other operating parameters is used to generate electromagnetic radiation, such as laser light, in a controlled, pulsed manner to be delivered through a light tube or pipe 12 to a projector. The light generating unit 10, such as comprising a laser console, will generate one or more pulsed energy sources, such as a pulsed laser light beam, having the operating parameters discussed above preselected and fixed so as to be unalterable by a medical care provider or other end user. It will be understood by those skilled in the art that the light tube 12 could be connected to a camera, projector, or the light transmitted by the light generating unit 10 could be directly transmitted to such projector, camera or the like without the need of a light tube or pipe or the like.

[Para 61] With reference now to FIG. 4, a schematic diagram is shown of a system for generating electromagnetic energy radiation, such as laser light, including SDM. The system, generally referred to by the reference number 20, includes a laser console 22, such as for example the 810 nm near infrared micropulsed diode laser in the preferred embodiment. The laser generates a laser light beam which may be passed through optics, such as an optical lens or mask, or a plurality of optical lenses and/or masks 24 as needed. The laser projector optics 24 pass the shaped light beam to a delivery device 26 for projecting the laser beam light onto the target tissue of the patient. It will be understood that the box labeled 26 can represent both the laser beam projector or delivery device as well as a viewing system/camera, or comprise two different components in use. The viewing system/camera 26 provides feedback to a display monitor 28, which may also include the necessary computerized hardware, data input and controls, etc. for manipulating the laser 22, the optics 24, and/or the projection/viewing components 26.

[Para 62] With reference now to FIG. 5, in one embodiment, a plurality of light beams are generated, each of which has parameters selected so that a target tissue temperature may be controllably raised to therapeutically treat the target tissue without destroying or permanently damaging the target tissue. This may be done, for example, by passing the laser light beam 30 through optics which diffract or otherwise generate a plurality of laser light beams from the single laser light beam 30 having the selected parameters. For example, the laser light beam 30 may be passed through a collimator lens 32 and then through a mask 34. In a particularly preferred embodiment, the mask 34 comprises a diffraction grating. The mask/diffraction grating 34 produces a geometric object, or more typically a geometric pattern of simultaneously produced multiple laser spots or other geometric objects. This is represented by the multiple laser light beams labeled with reference number 36. Alternatively, the multiple laser spots may be generated by a plurality of fiber optic waveguides. Either method of generating laser spots allows for the creation of a very large number of laser spots simultaneously over a very wide treatment field. In fact, a very high number of laser spots, perhaps numbering in the hundreds even thousands or more could be simultaneously generated to cover a given area of the target tissue, or possibly even the entirety of the target tissue. A wide array of simultaneously applied small separated laser spot appli-cations may be desirable as such avoids certain disadvan-tages and treatment risks known to be associated with large laser spot applications.

[Para 63] Using optical features with a feature size on par with the wavelength of the laser employed, for example using a diffraction grating, it is possible to take advantage of quantum mechanical effects which permits simultaneous application of a very large number of laser spots for a very large target area. The individual spots produced by such diffraction gratings are all of a similar optical geometry to the input beam, with minimal power variation for each spot. The result is a plurality of laser spots with adequate irradiance to produce harmless yet effective treatment application, simultaneously over a large target area. The present invention also contemplates the use of other geometric objects and patterns generated by other diffractive optical elements.

[Para 64] The laser light passing through the mask 34 diffracts, producing a periodic pattern a distance away from the mask 34, shown by the laser beams labeled 36 in FIG. 21. The single laser beam 30 has thus been formed into hundreds or even thousands of individual laser beams 36 so as to create the desired pattern of spots or other geometric objects. These laser beams 36 may be passed through additional lenses, collimators, etc. 38 and 40 in order to convey the laser beams and form the desired pattern. Such additional lenses, collimators, etc. 38 and 40 can further transform and redirect the laser beams 36 as needed.

[Para 65] Arbitrary patterns can be constructed by controlling the shape, spacing and pattern of the optical mask 34. The pattern and exposure spots can be created and modified arbitrarily as desired according to application requirements by experts in the field of optical engineering. Photolithographic techniques, especially those developed in the field of semiconductor manufacturing, can be used to create the simultaneous geometric pattern of spots or other objects.

[Para 66] The present invention can use a multitude of simultaneously generated therapeutic light beams or spots, such as numbering in the dozens or even hundreds, as the parameters and methodology of the present invention create therapeutically effective yet non-destructive and non-permanently damaging treatment. Although hundreds or even thousands of simultaneous laser spots could be generated and created and formed into patterns to be simultaneously applied to the tissue, due to the requirements of not overheating the tissue, there are constraints on the number of treatment spots or beams which can be simultaneously used in accordance with the present invention. Each individual laser beam or spot requires a minimum average power over a train duration to be effective. However, at the same time, tissue cannot exceed certain temperature rises without becoming damaged. For example, using an 810 nm wavelength laser, the number of simultaneous spots generated and used could number from as few as 1 and up to approximately 100 when a 0.04 (4%) duty cycle and a total train duration of 0.3 seconds (300 milliseconds) is used. The water absorption increases as the wavelength is increased. For shorter wave-lengths, e.g., 577 nm, the laser power can be lower. For example, at 577 nm, the power can be lowered by a factor of 4 for the invention to be effective. Accordingly, there can be as few as a single laser spot or up to approximately 400 laser spots when using the 577 nm wavelength laser light, while still not harming or damaging the tissue.

[Para 67] Typically, the system of the present invention incorporates a guidance system to ensure complete and total retinal treatment with retinal photostimulation of the entire retina, or the selected portion of the retina. Fixation/tracking/registration systems consisting of a fixation target, tracking mechanism, and linked to system operation can be incorporated into the present invention. The geometric pattern of simultaneous laser spots may be sequentially offset so as to achieve con-fluent and complete treatment of the surface.

[Para 68] This can be done in a controlled manner using an optical scanning mechanism 50. FIGS. 6 and 7 illustrate an optical scanning mechanism 50 in the form of a MEMS mirror, having a base 52 with electronically actuated controllers 54 and 56 which serve to tilt and pan the mirror 58 as electricity is applied and removed thereto. Applying electricity to the controller 54 and 56 causes the mirror 58 to move, and thus the simultaneous pattern of laser spots or other geometric objects reflected thereon to move accordingly on the retina of the patient. This can be done, for example, in an automated fashion using electronic software program to adjust the optical scanning mechanism 50 until complete coverage of the retina, or at least the portion of the retina desired to be treated, is exposed to the phototherapy. The optical scanning mechanism may also be a small beam diameter scanning galvo mirror system, or similar system, such as that distributed by Thorlabs. Such a system is capable of scanning the lasers in the desired offsetting pattern.

[Para 69] The pattern of spots are offset at each exposure so as to create space between the immediately previous expo-sure to allow heat dissipation and prevent the possibility of heat damage or tissue destruction. Thus, as illustrated in FIG. 8, the pattern, illustrated for exemplary purposes as a grid of sixteen spots, is offset each exposure such that the laser spots occupy a different space than previous exposures. It will be understood that the diagrammatic use of circles or empty dots as well as filled dots are for diagrammatic purposes only to illustrate previous and subsequent exposures of the pattern of spots to the area, in accordance with the present invention. The spacing of the laser spots prevents overheating and damage to the tissue. It will be understood that this occurs until the entire target tissue to be treated has received phototherapy, or until the desired effect is attained. This can be done, for example, by applying electrostatic torque to a micromachined mirror, as illustrated in FIGS. 6 and 7. By combining the use of small laser spots separated by exposure free areas, prevents heat accumulation, and grids with a large number of spots per side, it is possible to atraumatically and invisibly treat large target areas with short exposure durations far more rapidly than is possible with current technologies.

[Para 70] By rapidly and sequentially repeating redirection or offsetting of the entire simultaneously applied grid array of spots or geometric objects, complete coverage of the target, can be achieved rapidly without thermal tissue injury. This offsetting can be determined algorithmically to ensure the fastest treatment time and least risk of damage due to thermal tissue, depending on laser parameters and desired application.

[Para 71] Furthermore, by virtue of the small apertures employed in the diffraction grating or mask, quantum mechanical behavior may be observed which allows for arbitrary distribution of the laser input energy. This would allow for the generation of any arbitrary geometric shapes or patterns, such as a plurality of spots in grid pattern, lines, or any other desired pattern. Other methods of generating geometric shapes or patterns, such as using multiple fiber optical fibers or microlenses, could also be used in the present invention. Time savings from the use of simultaneous projection of geometric shapes or patterns permits the treatment fields of novel size, such as the 1.2 cm² area to accomplish whole-retinal treatment, in a single clinical setting or treatment session. While the generation and application of a plurality of laser light beams to simultaneously create a corresponding plurality of target tissue treatment spots has its advantages, it will be understood that the present invention could be utilized with only a single treatment laser light beam which is controllably moved over a desired treatment area of the retina.

[Para 72] With reference now to FIG. 9, instead of a geometric pattern of small laser spots, the present invention contemplates use of other geometric objects or patterns. For example, a single line 60 of laser light, formed by the continuously or by means of a series of closely spaced spots, can be created. An offsetting optical scanning mechanism can be used to sequentially scan the line over an area, illustrated by the downward arrow in FIG. 9.

[Para 73] With reference now to FIG. 10, the same geometric object of a line 60 can be rotated, as illustrated by the arrows, so as to create a circular field of phototherapy. The potential negative of this approach, however, is that the central area will be repeatedly exposed, and could reach unacceptable temperatures. This could be overcome, however, by increasing the time between exposures, or creating a gap in the line such that the central area is not exposed.

[Para 74] The field of photobiology reveals that different biologic effects may be achieved by exposing target tissues to lasers of different wavelengths. The same may also be achieved by consecutively applying multiple lasers of either different or the same wavelength in sequence with variable time periods of separation and/or with different irradiant energies. The present invention anticipates the use of multiple laser, light or radiant wavelengths (or modes) applied simultaneously or in sequence to maximize or customize the desired treatment effects. This method also minimizes potential detrimental effects. The optical methods and systems illustrated and described above provide simultaneous or sequential application of multiple wavelengths.

[Para 75] FIG. 11 illustrates diagrammatically a system which couples multiple treatment light sources into the pattern-generating optical subassembly described above. Specifically, this system 20′ is similar to the system 20 described in FIG. 4 above. The primary differences between the alternate system 20′ and the earlier described system 20 is the inclusion of a plurality of laser consoles, the outputs of which are each fed into a fiber coupler 42. Each laser console may supply a laser light beam having different parameters, such as of a different wavelength. The fiber coupler produces a single output that is passed into the laser projector optics 24 as described in the earlier system. The coupling of the plurality of laser consoles 22 into a single optical fiber is achieved with a fiber coupler 42 as is known in the art. Other known mechanisms for combining multiple light sources are available and may be used to replace the fiber coupler described herein.

[Para 76] In this system 20′ the multiple light sources 22 follow a similar path as described in the earlier system 20, i.e., collimated, diffracted, recollimated, and directed to the projector device and/or tissue. In this alternate system 20′ the diffractive element must function differently than described earlier depending upon the wavelength of light passing through, which results in a slightly varying pattern. The variation is linear with the wavelength of the light source being diffracted. In general, the difference in the diffraction angles is small enough that the different, overlapping patterns may be directed along the same optical path through the projector device 26 to the tissue for treatment.

[Para 77] Since the resulting pattern will vary slightly for each wavelength, a sequential offsetting to achieve complete coverage will be different for each wavelength. This sequential offsetting can be accomplished in two modes. In the first mode, all wavelengths of light are applied simultaneously without identical coverage. An offsetting steering pattern to achieve complete coverage for one of the multiple wavelengths is used. Thus, while the light of the selected wavelength achieves complete coverage of the tissue, the application of the other wavelengths achieves either incomplete or overlapping coverage of the tissue. The second mode sequentially applies each light source of a varying wavelength with the proper steering pattern to achieve complete coverage of the tissue for that particular wavelength. This mode excludes the possibility of simultaneous treatment using multiple wavelengths, but allows the optical method to achieve identical coverage for each wavelength. This avoids either incomplete or overlapping coverage for any of the optical wavelengths.

[Para 78] These modes may also be mixed and matched. For example, two wavelengths may be applied simultaneously with one wavelength achieving complete coverage and the other achieving incomplete or overlapping coverage, followed by a third wavelength applied sequentially and achieving complete coverage.

[Para 79] FIG. 12 illustrates diagrammatically yet another alternate embodiment of the inventive system 20″. This system 20″ is configured generally the same as the system 20 depicted in FIG. 4. The main difference resides in the inclusion of multiple pattern-generating subassembly channels tuned to a specific wavelength of the light source. Multiple laser consoles 22 are arranged in parallel with each one leading directly into its own laser projector optics 24. Similar to the above, the operating parameters of the laser console 22 and parameters of the resultant laser treatment light beams will be preselected and fixed so as to be unalterable by a medical care provider or other end user, per the present invention. The parameters of one or more or even each of the laser treatment light beams could be different from one another, but still preselected and fixed so as to be unalterable.

[Para 80] The laser projector optics of each channel 44a, 44b, 44c comprise a collimator 32, mask or diffraction grating 34 and recollimators 38, 40 as described in connection with FIG. 11 above--the entire set of optics tuned for the specific wavelength generated by the corresponding laser console 22. The output from each set of optics 24 is then directed to a beam splitter 46 for combination with the other wavelengths. It is known by those skilled in the art that a beam splitter used in reverse can be used to combine multiple beams of light into a single output. The combined channel output from the final beam splitter 46c is then directed through the projector device 26.

[Para 81] In this system 20″ the optical elements for each channel are tuned to produce the exact specified pattern for that channel's wavelength.

Consequently, when all channels are combined and properly aligned a single steering pattern may be used to achieve complete coverage of the tissue for all wavelengths. The system 20″ may use as many channels 44a, 44b, 44c, etc. and beam splitters 46a, 46b, 46c, etc. as there are wavelengths of light being used in the treatment.

[Para 82] Implementation of the system 20″ may take advantage of different symmetries to reduce the number of alignment constraints. For example, the proposed grid patterns are periodic in two dimensions and steered in two dimensions to achieve complete coverage. As a result, if the patterns for each channel are identical as specified, the actual pattern of each channel would not need to be aligned for the same steering pattern to achieve complete coverage for all wavelengths. Each channel would only need to be aligned optically to achieve an efficient combination.

[Para 83] In system 20″, each channel begins with a light source 22, which could be from an optical fiber as in other embodiments of the pattern-generating subassembly. This light source 22 is directed to the optical assembly 24 for collimation, diffraction, recollimation and directed into the beam splitter which combines the channel with the main output.

[Para 84] It will be understood that the laser light generating systems illustrated in FIGS. 4-12 are exemplary. Other devices and systems can be utilized to generate a source of SDM laser light which can be operably passed through to a projector device.

[Para 85] The proposed treatment with a train of electromagnetic pulses has two major advantages over earlier treatments that incorporate a single short or sustained (long) pulse. First, the short (preferably subsecond) individual pulses in the train activate cellular reset mechanisms like HSP activation with larger reaction rate constants than those operating at longer (minute or hour) time scales. Secondly, the repeated pulses in the treatment provide large thermal spikes (on the order of 10,000) that allow the cell's repair system to more rapidly surmount the activation energy barrier that separates a dysfunctional cellular state from the desired functional state. The net result is a “lowered therapeutic threshold” in the sense that a lower applied average power and total applied energy can be used to achieve the desired treatment goal.

[Para 86] Power limitations in current micropulsed diode lasers require fairly long exposure duration. The longer the exposure, the more important the center-spot heat dissipating ability toward the unexposed tissue at the margins of the laser spot. Thus, the micropulsed laser light beam of an 810 nm diode laser should have an exposure envelope duration of 500 milliseconds or less, and preferably approximately 300 milliseconds. Of course, if micropulsed diode lasers become more powerful, the exposure duration should be lessened accordingly.

[Para 87] Aside from power limitations, another parameter of the present invention is the duty cycle, or the frequency of the train of micropulses, or the length of the thermal relaxation time between consecutive pulses. It has been found that the use of a 10% duty cycle or higher adjusted to deliver micropulsed laser at similar irradiance at similar MPE levels significantly increase the risk of lethal cell injury. However, duty cycles of less than 10%, and preferably 5% or less demonstrate adequate thermal rise and treatment at the level of the MPE cell to stimulate a biological response, but remain below the level expected to produce lethal cell injury. The lower the duty cycle, however, the exposure envelope duration increases, and in some instances can exceed 500 milliseconds.

[Para 88] Each micropulse lasts a fraction of a millisecond, typically between 50 microseconds to 100 microseconds in duration. Thus, for the exposure envelope duration of 300-500 milliseconds, and at a duty cycle of less than 5%, there is a significant amount of time between micropulses to allow the thermal relaxation time between consecutive pulses. Typically, a delay of between 1 and 3 milliseconds, and preferably approximately 2 milliseconds, of thermal relaxation time is needed between consecutive pulses. For adequate treatment, the cells are typically exposed or hit between 50-200 times, and preferably between 75-150 at each location, and with the 1-3 milliseconds of relaxation or interval time, the total time in accordance with the embodiments described above to treat a given area which is being exposed to the laser spots is usually less than one second, such as between 100 milliseconds and 600 milliseconds on average. The thermal relaxation time is required so as not to overheat the cells within that location or spot and so as to prevent the cells from being damaged or destroyed.

[Para 89] Adjacent exposure areas must be separated by at least a predetermined minimum distance to avoid thermal tissue damage. Such distance is at least 0.5 diameter away from the immediately preceding treated location or area, and more preferably between 1-2 diameters away. Such spacing relates to the actually treated locations in a previous exposure area. It is contemplated by the present invention that a relatively large area may actually include multiple exposure areas therein which are offset.

[Para 90] Although the present invention is described for use in connection with a micropulsed laser, theoretically a continuous wave laser could potentially be used instead of a micropulsed laser. However, with the continuous wave laser, there is concern of overheating as the laser is moved from location to location in that the laser does not stop and there could be heat leakage and overheating between treatment areas. Thus, while it is theoretically possible to use a continuous wave laser, in practice it is not ideal and the micropulsed laser is preferred.

[Para 91] As mentioned above, the controlled manner of applying energy to the target tissue is intended to raise the temperature of the target tissue to therapeutically treat the target tissue without destroying or permanently damaging the target tissue. It is believed that such heating activates HSPs and that the thermally activated HSPs work to reset the diseased tissue to a healthy condition, such as by removing and/or repairing damaged proteins. It is believed by the inventors that maximizing such HSP activation improves the therapeutic effect on the targeted tissue. As such, understanding the behavior and activation of HSPs and HSP system species, their generation and activation, temperature ranges for activating HSPs and time frames of the HSP activation or generation and deactivation can be utilized to optimize the heat treatment of the biological target tissue.

[Para 92] As mentioned above, the target tissue is heated by the pulsed energy for a short period of time, such as ten seconds or less, and typically less than one second, such as between 100 milliseconds and 600 milliseconds. The time that the energy is actually applied to the target tissue is typically much less than this in order to provide intervals of time for heat relaxation so that the target tissue does not overheat and become damaged or destroyed. For example, as mentioned above, laser light pulses may last on the order of microseconds with several milliseconds of intervals of relaxed time.

[Para 93] Thus, understanding the sub-second behaviors of HSPs can be important to the present invention. The thermal activation of the HSPs in SDM is typically described by an associated Arrhenius integral,

Ω=∫dt A exp[−E/k₈T(t)] [1]

where the integral is over the treatment time and

[Para 94] A is the Arrhenius rate constant for HSP activation

[Para 95] E is the activation energy

[Para 96] T(t) is the temperature of the thin RPE layer, including the laser-induced temperature rise

[Para 97] The laser-induced temperature rise—and therefore the activation Arrhenius integral—depends on both the treatment parameters (e.g., laser power, duty cycle, total train duration) and on the RPE properties (e.g., absorption coefficients, density of HSPs). It has been found clinically that effective SDM treatment is obtained when the Arrhenius integrals is of the order of unity.

[Para 98] The Arrhenius integral formalism only takes into account a forward reaction, i.e. only the HSP activation reaction): It does not take into account any reverse reactions in which activated HSPs are returned to their inactivated states. For the typical subsecond durations of SDM treatments, this appears to be quite adequate. However, for longer periods of time (e.g. a minute or longer), this formalism is not a good approximation: At these longer times, a whole series of reactions occurs resulting in much smaller effective HSP activation rates. This is the case during the proposed minute or so intervals between SDM applications in the present invention disclosure.

[Para 99] In the published literature, the production and destruction of heat shock proteins (HSPs) in cells over longer durations is usually described by a collection of 9-13 simultaneous mass-balance differential equations that describe the behavior of the various molecular species involved in the life cycle of an HSP molecule. These simultaneous equations are usually solved by computer to show the behavior in time of the HSPs and the other species after the temperature has been suddenly raised.

These equations are all conservation equations based on the reactions of the various molecular species involved in the activity of HSPs. To describe the behavior of the HSPs in the minute or so intervals between repeated applications of SDM, we shall use the equations described in M. Rybinski, Z. Szymanska, S. Lasota, A. Gambin (2013) Modeling the efficacy of hyperthermia treatment. Journal of the Royal Society Interface 10, No. 88, 20130527 (Rybinski et al (2013)). The species considered in Rybinski et al (2013) are shown in Table 1.

TABLE 1
HSP system species in Rybinski et al (2013) description:

| | |
|---|---|
| HSP | ubiquitous heat shock protein of molecular |
| | weight 70 Da (in free, activated state) |
| HSF | heat shock (transcription) factor that has no |
| | DNA binding capability |
| HSF₃ | (trimer) heat shock factor capable of binding |
| | to DNA, formed from HSF |
| HSE | heat shock element, a DNA site that initiates |
| | transcription of HSP when bound to HSF₃ |
| mRNA | messenger RNA molecule for producing HSP |
| S | substrate for HSP binding: a damaged protein |
| P | properly folded protein |
| HSP. | a complex of HSP bound to HSF |
| HSF | (unactivated HSPs) |
| HSF₃. | a complex of HSF₃ bound to HSE, that |
| HSE | induces transcription and the creation of a |
| | new HSP mRNA molecule |
| HSP. | a complex of HSP attached to damaged |
| S | protein (HSP actively repairing the |
| | protein) |

[Para 101] The coupled simultaneous mass conservation equations for these 10 species are summarized below as eqs. [2]-[11]:

d[HSP]/dt=(I₁+k₁₀)[HSPS]+I₂[HSPHSF]+k₄[mRNA]−k₁[S][HSP]−k₂[HSP][HSF]−I₃[HSP][HSF₃]−k₉[HSP] [2]

d{HSF]/dt=I₂[HSPHSF]+2I₃[HSP][HSF₃]+k₆[HSPHSF][S]−k₂[HSP][HSF]−3k₃[HSF]³−I₆[HSPS][HSF] [3]

d[S]/dt=k₁₁{[P]+I₁[HSPS]+I₆[SPS][HSF]−k₁[S][HSP]−k₆[HSPHSF][S] [4]

d[HSPHSF]/dt=k₂[HSP][HSF]+I₆[HSPS][HSF]+I₃[HSP][HSF₃]−I₂[HSPHSF]−k₆[HSPHSF][S] [5]

d[HSPS]/dt=k₁[S][HSP]+k₆[HSPHSF][S]−(I₁+k₁₀)[HSPS]−I₆[HSPS][HSF] [6]

d[HSF₃]/dt=k₃[HSF]³+I₇[HSF₃][HSE]−I₃[HSP][HSF₃]−k₇[HSF₃][HSE] [7]

d[HSE]/dt=I₇[HSF₃][HSE]−k₇[HSF₃][HSE] [8]

d[HSF₃HSE]/dt=k₇[HSF₃][HSE]−I₇[HSF₃][HSE] [9]

d[mRNA]/dt=k₈[HSF₃HSE]−k₅[mRNA] [10]

d[P]/dt=k₁₀[HSPS]−k₁₁[P] [11]

[Para 102] In these expressions, [ ] denotes the cellular concentration of the quantity inside the bracket. For Rybinski et al (2013), the initial concentrations at the equilibrium temperature of 310K are given in Table 2.0

TABLE 2
Initial values of species at 310K for a typical cell in
arbitrary units [Rybinski et al (2013)]. The arbitrary
units are chosen by Rybinski et al for computational
convenience: to make the quantities of interest in
the range of 0.01-10.

| | |
|---|---|
| [HSP(0)] | 0.308649 |
| [HSF(0)] | 0.150836 |
| [S(0)] | 0.113457 |
| [HSPHSF(0)] | 2.58799 |
| [HSPS(0)] | 1.12631 |
| [HSF₃(0)] | 0.0444747 |
| [HSE(0)] | 0.957419 |
| [HSF₃HSE(0)] | 0.0425809 |
| [mRNA(0)] | 0.114641 |
| [P(0)] | 8.76023 |

[Para 1 04] The Rybinski et al (2013) rate constants are shown in Table 3.

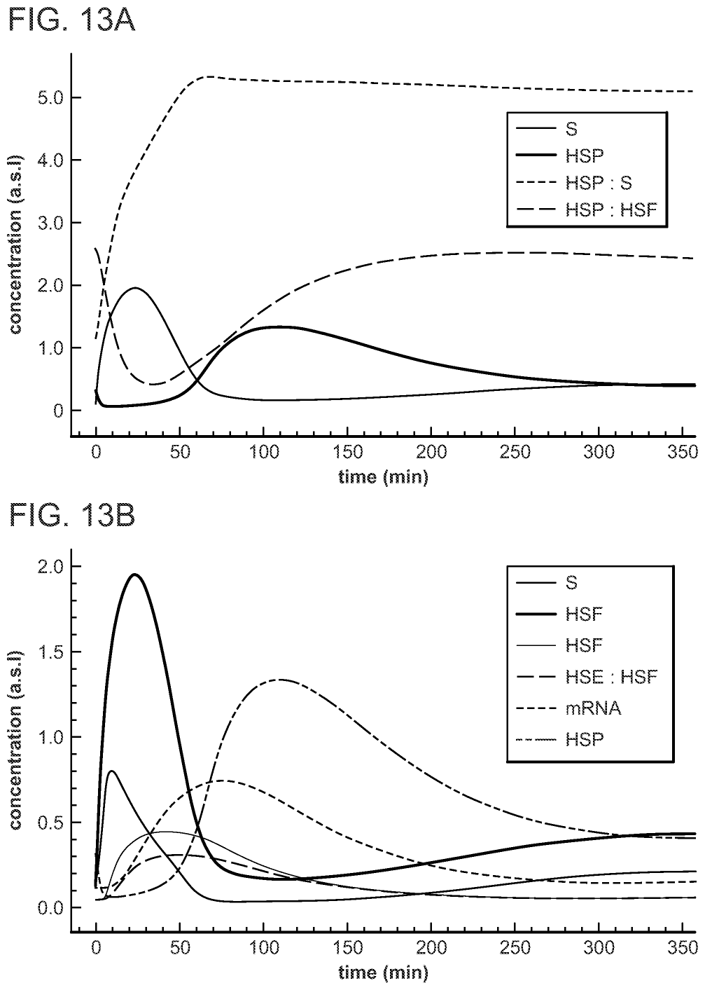

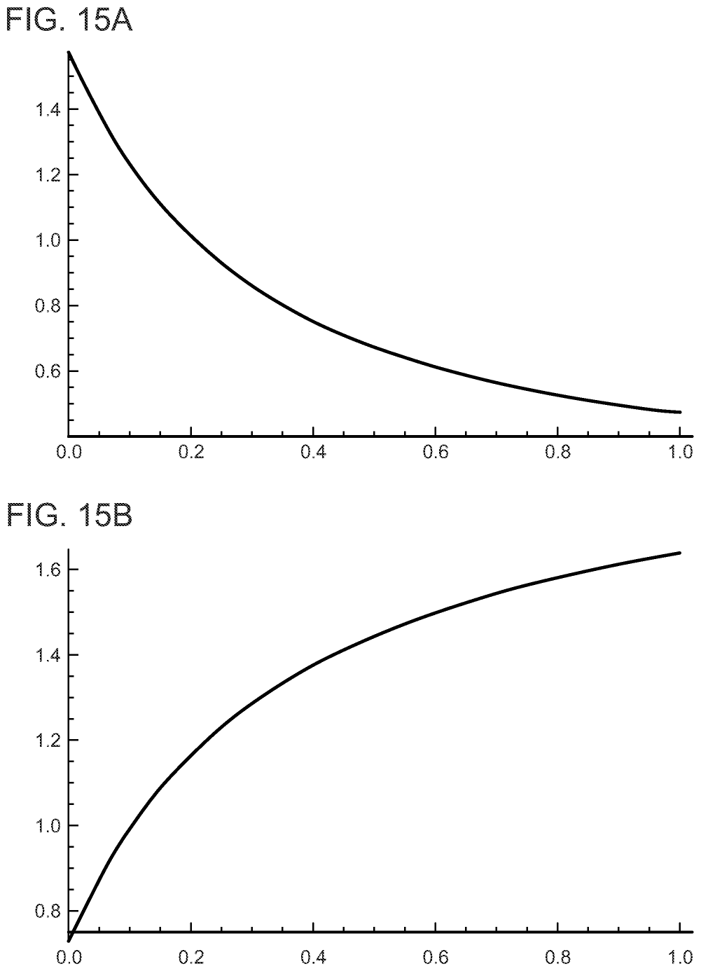

TABLE 3
Rybinski et al (2013) rate constants giving
rates in min⁻¹ for the arbitrary concen-
tration units of the previous table.
l₁ = 0.0175
k₁ = 1.47
l₂ = 0.0175
k₂ = 1.47
l₃ = 0.020125
k₃ = 0.0805
k₄ = 0.1225
k₅ = 0.0455
k₆ = 0.0805
l₆ = 0.00126
k₇ = 0.1225
l₇ = 0.1225
k₈ = 0.1225
k₉ = 0.0455
k₁₀ = 0.049
k₁₁ = 0.00563271

[Para 106] The initial concentration values of Table 2 and the rate constants of Table 3 were determined by Rybinski et al (2013) to correspond to experimental data on overall HSP system behavior when the temperature was increased on the order of 5° C. for several (e.g. 350) minutes.

[Para 107] Note that the initial concentration of HSPs is 100×0.308649/(8.76023+0.113457+1.12631)}=3.09% of the total number of proteins present in the cell.

[Para 108] Although the rate constants of Table 3 are used by Rybinski et al for T=310+5+31 5K, it is likely that very similar rate constants exist at other temperatures. In this connection, the qualitative behavior of the simulations is similar for a large range of parameters. For convenience, we shall assume that the values of the rate constants in Table 3 are a good approximation for the values at the equilibrium temperature of T=310K.

[Para 109] The behavior of the different components in the Rybinski et al cell is displayed in FIG. 13 for 350 minutes for the situation where the temperature is suddenly increased 5K at t=0 from an ambient 310K.

[Para 110] With continuing reference to FIG. 13, the behavior of HSP cellular system components during 350 minutes following a sudden increase in temperature from 37° C. to 42° C. is shown.

[Para 111] Here, the concentrations of the components are presented in computationally convenient arbitrary units. S denotes denatured or damaged proteins that are as yet unaffected by HSPs; HSP denotes free (activated) heat shock proteins; HSP:S denotes activated HSPs that are attached to the damaged proteins and performing repair; HSP:HSF denotes (inactive) HSPs that are attached to heat shock factor monomers; HSF denotes a monomer of heat shock factor; HSF₃ denotes a trimer of heat shock factor that can penetrate the nuclear membrane to interact with a heat shock element on the DNA molecule; HSE:HSF₃ denotes a trimer of heat shock factor attached to a heat shock element on the DNA molecule that initiates transcription of a new mRNA molecule; mRNA denotes the messenger RNA molecule that results from the HSE:HSF₃, and that leads to the production of a new (activated) HSP molecule in the cell's cytoplasm.

[Para 112] FIG. 13 shows that initially the concentration of activated HSPs is the result of release of HSPs sequestered in the molecules HSPHSF in the cytoplasm, with the creation of new HSPs from the cell nucleus via mRNA not occurring until 60 minutes after the temperature rise occurs. FIG. 13 also shows that the activated HSPs are very rapidly attached to damaged proteins to begin their repair work. For the cell depicted, the sudden rise in temperature also results in a temporary rise in damaged protein concentration, with the peak in the damaged protein concentration occurring about 30 minutes after the temperature increase.

[Para 113] FIG. 13 shows what the Rybinski et al equations predict for the variation of the 10 different species over a period of 350 minutes. However, the present invention is concerned with SDM application is on the variation of the species over the much shorter O(minute) interval between two applications of SDM at any single retinal locus. It will be understood that the preferred embodiment of SDM in the form of laser light treatment is analyzed and described, but it is applicable to other sources of energy as well.

[Para 114] With reference now to FIGS. 14A-14H, the behavior of HSP cellular system components during the first minute following a sudden increase in temperature from 37° C. to 42° C. using the Rybinski et al. (2013) equations with the initial values and rate constants of Tables 2 and 3 are shown. The abscissa denotes time in minutes, and the ordinate shows concentration in the same arbitrary units as in FIG. 15.

[Para 115] FIG. 15 shows that the nuclear source of HSPs plays virtually no role during a 1 minute period, and that the main source of new HSPs in the cytoplasm arises from the release of sequestered HSPs from the reservoir of HSPHSF molecules. It also shows that a good fraction of the newly activated HSPs attach themselves to damaged proteins to begin the repair process.

[Para 116] The initial concentrations in Table 2 are not the equilibrium values of the species, i.e. they do not give d[ . . . ]/dt=0, as evidenced by the curves in FIGS. 13 and 14. The equilibrium values that give d[ . . . ]/dt=0 corresponding to the rate constants of Table 3 are found to be those listed in Table 4.

TABLE 4
Equilibrium values of species in arbitrary units
[Rybinski et al (2013)] corresponding to the rate
constants of Table 3. The arbitary units are those
chosen by Rybinski et al for computational
convenience: to make the quantities of interest
in the range of 0.01-10.

| | |
|---|---|
| [HSP(equil)] | 0.315343 |
| [HSF(equil)] | 0.255145 |
| [S(equil)] | 0.542375 |
| [HSPHSF(equil)] | 1.982248 |
| [HSPS(equil)] | 5.05777 |
| [HSF₃(equil)] | 0.210688 |
| [HSE(equil)] | 0.206488 |
| [HSF₃HSE(equil)] | 0.643504 |
| [mRNA(equil)] | 0.1171274 |
| [P(equil)] | 4.39986 |

[Para 118] Note that the equilibrium concentration of HSPs is 100.times.{0.315343/(4.39986+5.05777+0.542375)}=3.15% of the total number of proteins present in the cell. This is comparable, but less than the anticipated 5%-10% total number of proteins found by other researchers. However, we have not attempted to adjust percentage upwards expecting that the general behavior will not be appreciably changed as indicated by other researchers.

[Para 119] The inventors have found that a first treatment to the target tissue may be performed by repeatedly applying the pulsed energy (e.g., SDM) to the target tissue over a period of time so as to controllably raise a temperature of the target tissue to therapeutically treat the target tissue without destroying or permanently damaging the target tissue. A “treatment” comprises the total number of applications of the pulsed energy to the target tissue over a given period of time, such as dozens or even hundreds of light or other energy applications to the target tissue over a short period of time, such as a period of less than ten seconds, and more typically a period of less than one second, such as 100 milliseconds to 600 milliseconds. This “treatment” controllably raises the temperature of the target tissue to activate the heat shock proteins and related components.

[Para 120] What has been found, however, is that if the application of the pulsed energy to the target tissue is halted for an interval of time, such as an interval of time that exceeds the first period of time comprising the “first treatment”, which may comprise several seconds to several minutes, such as three seconds to three minutes or more preferably ten seconds to ninety seconds, and then a second treatment is performed on the target tissue after the interval of time within a single treatment session or office visit, wherein the second treatment also entails repeatedly reapplying the pulsed energy to the target tissue so as to controllably raise the temperature of the target tissue to therapeutically treat the target tissue without destroying or permanently damaging the target tissue, the amount of activated HSPs and related components in the cells of the target tissue is increased resulting in a more effective overall treatment of the biological tissue. In other words, the first treatment creates a level of heat shock protein activation of the target tissue, and the second treatment increases the level of heat shock protein activation in the target tissue above the level due to the first treatment. Thus, performing multiple treatments to the target tissue of the patient within a single treatment session or office visit enhances the overall treatment of the biological tissue so long as the second or additional treatments are performed after an interval of time which does not exceed several minute but which is of sufficient length so as to allow temperature relaxation so as not to damage or destroy the target tissue.

[Para 121] This technique may be referred to herein as “stair-stepping” in that the levels of activated HSP production increase with the subsequent treatment or treatments within the same office visit treatment session. This “stair-stepping” technique may be described by a combination of the Arrhenius integral approach for subsecond phenomena with the Rybinski et al. (2013) treatment of intervals between repeated subsecond applications of the SDM or other pulsed energy.

[Para 122] For the proposed stair-stepping SDM (repetitive SDM applications) proposed in this invention disclosure, there are some important differences from the situation depicted in FIG. 13:

[Para 123] SDM can be applied prophylactically to a healthy cell, but oftentimes SDM will be applied to a diseased cell. In that case, the initial concentration of damaged proteins [S(0)] can be larger than given in Table 4. We shall not attempt to account for this, assuming that the qualitative behavior will not be changed.

[Para 124] The duration of a single SDM application is only subseconds, rather than the minutes shown in FIG. 13. The Rybinski et al rate constants are much smaller than the Arrhenius constants: the latter give Arrhenius integrals of the order of unity for subsecond durations, whereas the Rybinski et al rate constants are too small to do that. This is an example of the different effective rate constants that exist when the time scales of interest are different: The Rybinski et al rate constants apply to phenomena occurring over minutes, whereas the Arrhenius rate constants apply to subsecond phenomena.

[Para 125] Accordingly, to analyze what happens in the proposed stair-stepping SDM technique for improving the efficacy of SDM, we shall combine the Arrhenius integral treatment appropriate for the subsecond phenomena with the Rybinski et al (201 3) treatment appropriate for the phenomena occurring over the order of a minute interval between repeated SDM applications:

[Para 126] SDM subsecond application described by Arrhenius integral formalism

[Para 127] Interval of O(minute) between SDM applications described by Rybinski et al (2013) equations

[Para 128] Specifically, we consider two successive applications of SDM, each SDM micropulse train having a subsecond duration.
For the short subsecond time scale, we assume that the unactivated HSP's that are the source of the activated (free) HSP's are all contained in the HSPHSF molecules in the cytoplasm. Accordingly, the first SDM application is taken to reduce the cytoplasmic reservoir of unactivated HSPs in the initial HSPHSF molecule population from
   [HSPHSF(equil)] to [HSPHSF(equil)]exp[−Ω],
and to increase the initial HSP molecular population from
   [HSP(equil)] to [HSP(equil)]+[HSPHSF(equil)](1-exp[−Ω])
as well as to increase the initial HSF molecular population from
   [HSF(equil)] to [HSF(equil)]+[HSPHSF(equil)](1-exp[−Ω])
The equilibrium concentrations of all of the other species will be assumed to remain the same after the first SDM application
The Rybinski et al equations are then used to calculate what happens to [HSP] and [HSPHSF] in the interval [symbol] t=O(minute) between the first SDM application and the second SDM application, with the initial values of HSP, HSF and HSPHSF after the first SDM application taken to be
   [HSP(SDM1)]=[HSP(equil)]+[HSPHSF(equil)](1 -exp[−Ω])
   [HSF(SDM1)]=[HSF(equil)]+[HSPHSF(equil)](1-exp[−Ω])
and
   [HSPHSF(SDM1)]=[HSPHSF(equil)]exp[−Ω]
For the second application of SDM after the interval [symbol] t, the values of [HSP], [HSF] and {HSPHSF] after the SDM will be taken to be
   [HSP(SDM2)]=[HSP( [symbol] t)]+[HSPHSF( [symbol] t)](1-exp[−Ω])
   [HSF(SDM2)]=[HSF( [symbol] t)]+[HSPHSF( [symbol] t)](1-exp[−Ω])
and
   [HSPHSF(SDM2)]=[HSPHSF( [symbol] t)]exp[−Ω]
where [HSP(Xt)], [HSF( [symbol] t)], and [HSPHSF( [symbol] t)] are the values determined from the Rybinski et al (2013) equations at the time [symbol] t.
Our present interest is in comparing [HSP[SDM2)] with [HSP[SDM1)], to see if the repeated application of SDM at an interval [symbol] t following the first application of SDM has resulted in more activated (free) HSP's in the cytoplasm. The ratio β( [symbol] t, Ω)=[HSP(SDM2)]/[HSP(SDM1)]={[{[HSP( [symbol] t)]+[HSPHSF( [symbol] t)](1-exp[−Ω)}/{ [HSP(0)]+[HSPHSF(0)](1-exp[−Ω])}

provides a direct measure of the improvement in the degree of HSP activation for a repeated application of SDM after an interval [symbol] t from the first SDM application.

[Para 129] The HSP and HSPHSF concentrations can vary quite a bit in the interval t between SDM applications.

[Para 130] FIGS. 15A and 15B illustrate the variation in the activated concentrations [HSP] and the unactivated HSP in the cytoplasmic reservoir [HSPHSF] during an interval [symbol] t=1 minute between SDM applications when the SDM Arrhenius integral Ω=1 and the equilibrium concentrations are as given in Table 4.

[Para 131] Although only a single repetition (one-step) is treated here, it is apparent that the procedure could be repeated to provide a multiple stair-stepping events as a means of improving the efficacy of SDM, or other therapeutic method involving activation of tissue HSPs.

[Para 132] Effects of varying the magnitude of the Arrhenius integral Ω and interval [symbol] t between two distinct treatments separated by an interval of time are shown by the following examples and results.

[Para 133] Nine examples generated with the procedure described above are presented in the following. All of the examples are of a treatment consisting of two SDM treatments, with the second occurring at a time [symbol] t following the first, and they explore:
The effect of different magnitude Arrhenius integrals Ω in the SDM treatments [Three different Ω's are considered: Ω=0.2,0.5 and 1.0]
The impact of varying the interval [symbol] t between the two SDM treatments [Three different [symbol] t's are considered: [symbol] t=15 sec., 30 sec., and 60 sec.

[Para 134] As indicated above, the activation Arrhenius integral Ω depends on both the treatment parameters (e.g., laser power, duty cycle, total train duration) and on the RPE properties (e.g., absorption coefficients, density of HSPs).

[Para 135] Table 5 below shows the effect of different Ω (Ω=0.2, 0.5, 1) on the HSP content of a cell when the interval between the two SDM treatments is [symbol] t=1 minute . Here the cell is taken to have the Rybinski et al (2013) equilibrium concentrations for the ten species involved, given in Table 4.

[Para 136] Table 5 shows four HSP concentrations (in the Rybinski et al arbitrary units) each corresponding to four different times:
Before the first SDM treatment: [HSP(equil)]
Immediately after the first SDM application: [HSP(SDM1)]
At the end of the interval [symbol] t following the first SDM treatment: [HSP( [symbol] t)]
Immediately after the second SDM treatment at [symbol] t: [HSP(SDM2)]
Also shown is the improvement factor over a single treatment: β=[HSP(SDM2)]/[HSP(SDM1)]

TABLE 5
HSP concentrations at the four times just described
in the text: Effect of varying the SDM Ω for two
SDM applications on a cell when the treatments
are separated by [symbol] t = 0.25 minutes = 15 seconds.

| | [HSP | [HSP | [HSP | [HSP | |
|---|---|---|---|---|---|
| | (equil)] | (SDM1)] | ( [symbol] t)] | (SDM2)] | β |
| Ω = 0.2 | 0.315 | 0.67 | 0.54 | 0.95 | 1.27 |
| Ω = 0.5 | 0.315 | 1.10 | 0.77 | 1.34 | 1.22 |
| Ω = 1.0 | 0.315 | 1.57 | 0.93 | 1.71 | 1.09 |

[Para 138] Table 6 is the same as Table 5, except that it is for an interval between SDM treatments of [symbol] t=0.5 minutes=30 seconds.

TABLE 6
HSP concentrations at the four times described
in the text: Effect of varying the SDM Ω for two
SDM treatments on a cell when the treatments
are separated by [symbol] t = 0.5 minutes = 30 seconds.

| | [HSP | [HSP | [HSP | [HSP | |
|---|---|---|---|---|---|
| | (equil)] | (SDM1)] | ( [symbol] t)] | (SDM2)] | β |
| Ω = 0.2 | 0.315 | 0.67 | 0.44 | 0.77 | 1.14 |
| Ω = 0.5 | 0.315 | 1.10 | 0.58 | 1.18 | 1.08 |
| Ω = 1.0 | 0.315 | 1.57 | 0.67 | 1.59 | 1.01 |

[Para 140] Table 7 is the same as the Tables 5 and 6, except that the treatments are separated by one minute, or sixty seconds.

TABLE 7
HSP concentrations at the four times just described
in the text: Effect of varying the SDM Ω for two
SDM treatments on a normal (healthy) cell when
the treatments are separated by [symbol] t = 1 minute =
60 seconds.

| | [HSP | [HSP | [HSP | [HSP | |
|---|---|---|---|---|---|
| | (equil)] | (SDM1)] | ( [symbol] t)] | (SDM2)] | β |
| Ω = 0.2 | 0.315 | 0.67 | 0.30 | 0.64 | 0.95 |
| Ω = 0.5 | 0.315 | 1.10 | 0.37 | 1.06 | 0.96 |
| Ω = 1.0 | 0.315 | 1.57 | 0.48 | 1.51 | 0.96 |

[Para 142] Tables 5-7 show that:
The first treatment of SDM increases [HSP] by a large factor for all three Ω's, although the increase is larger the larger Ω. Although not displayed explicitly in the tables, the increase in [HSP] comes at the expense of the cytoplasmic reservoir of sequestered (unactivated) HSP's: [HSPHSF(SDM1)] is much smaller than [HSPHSF(equil)]
[HSP] decreases appreciably in the interval [symbol] t between the two SDM treatments, with the decrease being larger the larger [symbol] t is. (The decrease in [HSP] is accompanied by an increase in both [HSPHSF]—as shown in FIG. 15 and in [HSPS] during the interval [symbol] t—indicating a rapid replenishment of the cytoplasmic reservoir of unactivated HSP's and a rapid attachment of HSP's to the damaged proteins.)
For [symbol] t less than 60 seconds, there is an improvement in the number of activated (free) HSP's in the cytoplasm for two SDM treatments rather than a single treatment.
The improvement increases as [symbol] t becomes smaller.
For [symbol] t becoming as large as 60 seconds, however, the ratio β=[HSP(SDM2)]/[HSP(SDM1)] becomes less than unity, indicating no improvement in two SDM treatments compared to a single SDM treatment although this result can vary depending on energy source parameters and tissue type that is treated.
The improvement for [symbol] t<60 seconds is larger the smaller the SDM Arrhenius integral Ω is.

[Para 143] The results for the improvement ratio β=[HSP(SDM2)]/[HSP(SDM1)]are summarized in FIG. 16, where the improvement ratio β=[HSP(SDM2)]/[HSP(SDM1)]vs. interval between SDM treatments [symbol] t (in seconds) for three values of the SDM Arrhenius integral Ω, and for the three values of the interval [symbol] t=15 sec, 30 sec, and 60 sec . The uppermost curve is for Ω=0.2; the middle curve is for Ω=0.5; and the bottom curve is for Ω=1.0. These results are for the Rybinski et al (2013) rate constants of Table 3 and the equilibrium species concentrations of Table 4. [Para 144] It should be appreciated that results of Tables 5-7 and FIG. 16 are for the Rybinski et al. (2013) rate constants of Table 3 and the equilibrium concentrations of Table 4. The actual concentrations and rate constants in a cell may differ from these values, and thus the number results in Tables 5-7 and FIG. 16 should be taken as representative rather than absolute. However, they are not anticipated to be significantly different. Thus, performing multiple intra-sessional treatments on a single target tissue location or area, such as a single retinal locus, with the second and subsequent treatments following the first after an interval anywhere from three seconds to three minutes, and preferably ten seconds to ninety seconds, should increase the activation of HSPs and related components and thus the efficacy of the overall treatment of the target tissue. The resulting “stair-stepping” effect achieves incremental increases in the number of heat shock proteins that are activated, enhancing the therapeutic effect of the treatment. However, if the interval of time between the first and subsequent treatments is too great, then the “stair-stepping” effect is lessened or not achieved.

[Para 145] The technique of the present invention is especially useful when the treatment parameters or tissue characteristics are such that the associated Arrhenius integral for activation is low, and when the interval between repeated applications is small, such as less than ninety seconds, and preferably less than a minute. Accordingly, such multiple treatments must be performed within the same treatment session, such as in a single office visit, where distinct treatments can have a window of interval of time between them so as to achieve the benefits of the technique of the present invention.

[Para 1 46] Although several embodiments have been described in detail for purposes of illustration, various modifications may be made without departing from the scope and spirit of the invention. Accordingly, the invention is not to be limited, except as by the appended claims.

What is claimed is:

1. A retinal phototherapy or photostimulation system, comprising:
a laser console generating at least one pulsed treatment beam, the at least one treatment beam having parameters of wavelength, power, pulse train duration and duty cycle to photostimulate or treat a retinal target tissue while not permanently damaging the retinal target tissue, wherein the parameters of the at least one treatment beam are fixed so as to not be alterable by a medical provider;
a projector or camera for projecting the at least one treatment beam onto at least a portion of a retina; and
a scanning mechanism for controllably directing the at least one treatment beam to treatment areas of the retina.

2. The system of claim 1, wherein the at least one treatment beam stimulates heat shock protein activation in the target tissue.

3. The system of claim 1, wherein the at least one treatment beam raises a target tissue temperature to a desired level while maintaining an average temperature rise of the target tissue over a period of time at or below a predetermined level so as to not permanently damage the target tissue.

4. The system of claim 2, wherein the at least one treatment beam raises a target tissue temperature no greater than eleven degrees Celsius to achieve a therapeutic or prophylactic effect, wherein the average temperature rise of the target tissue over several minutes is maintained at or below a predetermined level so as to not permanently damage the target tissue.

5. The system of claim 4, wherein the at least one treatment beam raises the target tissue temperature between six degrees Celsius to eleven degrees Celsius at least during application of the pulsed energy source to the target tissue while maintaining the average temperature rise of the target tissue over several minutes at one degree Celsius or less.

6. The system of claim 5, wherein the average temperature of the target tissue is maintained at one degree Celsius or less over a six minute period of time.

7. The system of claim 1, wherein the at least one treatment beam is applied to a plurality of target tissue areas, and wherein adjacent target tissue areas are separated to avoid thermal tissue damage.

8. The system of claim 1, wherein the at least one treatment beam has a wavelength between 530 nm to 1300 nm, a duty cycle of less than 10%, and a pulse train duration between 0.1 and 0.6 seconds.

9. The system of claim 8, wherein the at least one treatment beam has a wavelength between 750 nm and 850 nm, a duty cycle of between 2% and 5%, and a pulse train duration of between 0.15 and 0.5 seconds.

10. The system of claim 1, wherein the laser console has a power output of between 0.5-3.0 watts.

11. The system of claim 1, wherein the at least one treatment beam creates a treatment spot on the retina having a size of 100-1,000 micrometers.

12. A retinal phototherapy or photostimulation system, comprising:
a laser console generating at least one pulsed treatment beam, the at least one treatment beam having parameters of wavelength, power, pulse train duration and duty cycle to photostimulate or treat a retinal target tissue while not permanently damaging the retinal target tissue, wherein the parameters of the at least one treatment beam are fixed so as to not be alterable by a medical provider;
a projector or camera for projecting the at least one treatment beam onto at least a portion of a retina; and
a scanning mechanism for controllably directing the at least one treatment beam to treatment areas of the retina;
wherein the at least one treatment beam raises a target tissue temperature between six degrees Celsius and eleven degrees Celsius to stimulate heat shock protein activation in the target tissue and achieve a therapeutic or prophylactic effect; and
wherein the average temperature rise of the target tissue over several minutes is maintained at or below one degree Celsius or less over a six minute time period so as to not permanently damage the target tissue.

13. The system of claim 12, wherein the at least one treatment beam is applied to a plurality of target tissue areas, and wherein adjacent target tissue areas are separated to avoid thermal tissue damage.

14. The system of claim 12, wherein the at least one treatment beam has a wavelength between 530 nm to 1300 nm, a duty cycle of less than 10%, and a pulse train duration between 0.1 and 0.6 seconds.

15. The system of claim 14, wherein the at least one treatment beam has a wavelength between 750 nm and 850 nm, a duty cycle of between 2% and 5%, and a pulse train duration of between 0.15 and 0.5 seconds.

16. The system of claim 12, wherein the laser console has a power output of between 0.5-3.0 watts.

17. The system of claim 12, wherein the at least one treatment beam creates a treatment spot on the retina having a size of 100-1,000 micrometers.

18. A retinal phototherapy or photostimulation system, comprising:
a laser console generating at least one pulsed treatment beam, the at least one treatment beam having parameters of wavelength, power, pulse train duration and duty cycle to photostimulate or treat a retinal target tissue while not permanently damaging the retinal target tissue, wherein the parameters of the at least one treatment beam are fixed so as to not be alterable by a medical provider;
a projector or camera for projecting the at least one treatment beam onto at least a portion of a retina; and
a scanning mechanism for controllably directing the at least one treatment beam to treatment areas of the retina wherein the at least one treatment beam has a wavelength between 530 nm to 1 300 nm, a duty cycle of less than 10%, and a pulse train duration between 0.1 and 0.6 seconds;
wherein the at least one treatment beam creates a treatment spot on the retina having a size of 100-1,000 micrometers; and
wherein the at least one treatment beam is applied to a plurality of target tissue areas, and wherein adjacent target tissue areas are separated to avoid thermal tissue damage.

19. The system of claim 18, wherein the at least one treatment beam has a wavelength between 750 nm and 850 nm, a duty cycle of between 2% and 5%, and a pulse train duration of between 0.15 and 0.5 seconds and the laser console has a power output of between 0.5-3.0 watts.

20. The system of claim 18, wherein the at least one treatment beam raises a target tissue temperature between six degrees Celsius and eleven degrees Celsius to stimulate heat shock protein activation in the target tissue and achieve a therapeutic or prophylactic effect; and wherein the average temperature rise of the target tissue over several minutes is maintained at or below one degree Celsius or less over a six minute time period so as to not permanently damage the target tissue
